SEQUENCE LISTING (22 sequences)

SEQ ID NO: 1 - DNA, 18 bp, Artificial Sequence
  antisense oligonucleotide
caaagatggactcgggag

SEQ ID NO: 2 - DNA, 18 bp, Artificial Sequence
  antisense oligonucleotide
gtcgctcttggtgctata

SEQ ID NO: 3 - DNA, 18 bp, Artificial Sequence
  antisense oligonucleotide
gctgctctgattgttggg

SEQ ID NO: 4 - DNA, 18 bp, Artificial Sequence
  antisense oligonucleotide
cctccactcttttctcag

SEQ ID NO: 5 - DNA, 18 bp, Artificial Sequence
  antisense oligonucleotide
aagcagacacccgagcag

SEQ ID NO: 6 - DNA, 18 bp, Artificial Sequence
  antisense oligonucleotide
ccctgagccatatcctgt

SEQ ID NO: 7 - DNA, 18 bp, Artificial Sequence
  antisense oligonucleotide
agaaccacagagcgacag

SEQ ID NO: 8 - DNA, 18 bp, Artificial Sequence
  antisense oligonucleotide
agtatgtctttctgtgtg

SEQ ID NO: 9 - DNA, 18 bp, Artificial Sequence
  antisense oligonucleotide
ctgtttccttcttctttg

SEQ ID NO: 10 - DNA, 18 bp, Artificial Sequence
  antisense oligonucleotide
tccttactcaccatttca

SEQ ID NO: 11 - DNA, 18 bp, Artificial Sequence
  antisense oligonucleotide
tgtttccttcttctttga

SEQ ID NO: 12 - DNA, 18 bp, Artificial Sequence
  antisense oligonucleotide
tactcaccatttcaggca

SEQ ID NO: 13 - DNA, 18 bp, Artificial Sequence
  antisense oligonucleotide
actcaccatttcaggcaa

SEQ ID NO: 14 - DNA, 18 bp, Artificial Sequence
  antisense oligonucleotide
agtatgtctttttgtatg

SEQ ID NO: 15 - DNA, 18 bp, Artificial Sequence
  antisense oligonucleotide
tgaagagttgtattagcc

SEQ ID NO: 16 - DNA, 18 bp, Artificial Sequence
  antisense oligonucleotide
actgccactctgattatt

SEQ ID NO: 17 - DNA, 18 bp, Artificial Sequence
  antisense oligonucleotide
tttgctcactgccactct

SEQ ID NO: 18 - DNA, 18 bp, Artificial Sequence
  antisense oligonucleotide
gtctttttgtatgctgag

SEQ ID NO: 19 - DNA, 18 bp, Artificial Sequence
  antisense oligonucleotide
agctaggcacgagagtga

SEQ ID NO: 20 - DNA, 18 bp, Artificial Sequence
  antisense oligonucleotide
tgctggcatgtgcgttgt

SEQ ID NO: 21 - DNA, 18 bp, Artificial Sequence
  antisense oligonucleotide
actgtccactcgcagttc

SEQ ID NO: 22 - DNA, 18 bp, Artificial Sequence
  antisense oligonucleotide
tttctggtatgcattgtg

(19) United States
(12) Patent Application Publication
(10) Pub. No.: US 2003/0186920 A1
(43) Pub. Date: Oct. 2, 2003

(54) Antisense oligonucleotide directed toward mammalian vegf receptor genes and uses thereof

(76) Inventor: Martin G. Sirois, Quebec (CA)

Correspondence Address: Marshall Gerstein & Borun, Sears Tower Suite 6300, 233 South Wacker Drive, Chicago, IL, 60606-6357, US

(21) Appl. No.: 10/399,019

(22) PCT Filed: Oct. 15, 2001
(86) PCT No.: PCT/CA01/01427

(51) Int. Cl. A61K 48/00; C07H 21/04; C12N 5/02

(52) U.S. Cl. 514/44; 435/375; 536/23.5

(57) ABSTRACT

The present invention provides antisense oligonucleotides that target the genes and mRNAs encoding mammalian VEGF receptors. Also provided are methods for designing and testing the antisense oligonucleotides. Such oligonucleotides can be used to reduce VEGF-induced inflammation and angiogenesis, for example, pathological angiogenesis, in mammals. Thus, the present invention also pertains to pharmaceutical compositions and formulations used in the treatment of mammals having a disease or disorder characterised by inflammation and/or pathological angiogenesis; including tumour growth and metastasis, ocular diseases (diabetic and perinatal hyperoxic retinopathies, age-related macular degeneration), arthritis, psoriasis and atherosclerosis.

### RELATED APPLICATIONS

This application is a continuation in part of U.S. application Ser. No. 09/687,239, filed Oct. 13, 2000, which is hereby incorporated in its entirety.

### FIELD OF THE INVENTION

The present invention pertains to the field of antisense oligonucleotides for mammalian VEGF receptor genes and their use as anti-angiogenics and/or anti-inflammatory agents.

### BACKGROUND

Angiogenesis is a process by which new capillary vessels sprout from pre-existing ones, and can be summarised as the culmination of i) increased endothelial cell permeability to plasma proteins; ii) transmigration of inflammatory cells into extracellular matrix; iii) synthesis and release of degrading matrix molecules; iv) release of growth factors; v) migration and proliferation of endothelial cells to distant sites; and vi) capillary tube formation and vascular wall remodelling. Physiological angiogenesis is a highly co-ordinated process that exclusively occurs in healthy individuals under specific conditions, such as during wound healing, ovulation and pregnancy. At other times, the vasculature is extremely stable, with very low rates of new blood vessels (Fan et al., (1995) Trends Phaimacol. Sci. 16:57-66).

Pathological angiogenesis is present in a number of disease states and biological conditions, including tumour growth and metastasis, ocular diseases (diabetic and perinatal hyperoxic retinopatlies, age-related macular degeneration), arthritis, psoriasis and atherosclerosis (Folkman et al., (1987) Science. 235:442-447; Ferrara and Davis-Smyth (1997) Endocinte Rev. 18:4-25; Moulton et al., (1999) Circulation 99:1726-1732; Ferrara (1999) J. Mol. Med. 77:527-543; Folkrman (1972) Ann. Surg. 175:409-416; Folkman and Shing (1992) J. Biol. Chem. 267:10931-10934). Thus, attempts have been made to develop methods of inhibiting pathological angiogenesis as potential therapeutic techniques.

Angiogenesis is the coordinated response to several factors including vascular endotlielial growth factor (VEGF), acidic and basic fibroblast growth factors (aFGF, bFGF), transforming growth factor-α and -β (TGF-α, TGF-β), hepatocyte growth factor (HGF), tumor-necrosis factor-α CMNF-α) angiogenin and others (Ferrara and Davis-Smyth (1997) Endocrine Rev. 18:4-25; Folkman and Shing (1992) J. Biol. Chem. 267:10931-10934;. Klagsbrun and D'Amore (1991) Annu. Rev. Physiol. 53:217-239). Growing evidence suggests that VEGF plays a pivotal role in the regulation of normal and pathophysiological angiogenesis (Folkman and Shing (1992) J. Biol. Chem. 267:10931-10934; Klagsbrun and D'Amore (1991) Annu. Rev. Physiol. 53:217-239; Breier and Risau (1996) Trends Cell Biol. 6:454-456; Ferrara (1993) Trends Cardiovasc. Med. 3:244-250). Similar to other growth factors VEGF can induce the proliferation and migration of endothelial cells, however, VEGF is the only growth factor known, to date, to have the ability to augment vascular permeability (Senger et al., (1983) Science. 219:983-985; Connolly et al., (1989) J. Clin. Invest. 84:1470-1478; Favard et al., 1991) Biol. Cell. 73:1-6).

The actions of VEGF and other family members are mediated by tyrosine kinase receptors, Flt-1 (VEGFR-1), Flk-1 (VEGFR-2), and Flt-4 (VEGFR-3), which are expressed almost exclusively on endothelial cells. VEGF is known to interact with both Flt-1 and Flk-1 in vivo, but there is no evidence of its interaction with Flt-4 (Neufeld et al., (1999) FASEB J. 13:9-22; Petrova et al., (1999) Exp. Cell Res. 253:117-130).

The importance of VEGF receptors in vascular development has been illustrated using gene-targeting approaches. Disruption of Flt-1, Flk-1, and Flt-4 leads to embryonic lethality (Petrova et al., (1999) Exp. Cell Res. 253:117-130). Flt-1 and Flk-1 are expressed predominantly in endothelial cells, and few other cell types express one or both receptors (Neufeld et al., (1999) FASEB J. 13:9-22; Petrova et al., (1999) Exp. Cell Res. 253:117-130; Jussila et al., (1998) Cancer Res. 58:1599-1604; de Vries et al., (1992) Science. 255:989-991; Tennan et al., (1992) Biochem. Biophys. Res. Commun. 34:1578-1586; Shibuya et al., (1990) Oncogene. 8:519-527; Quinn et al., (1993) Proc. Natl. Acad. Sci. U.S.A. 90:7533-7537). Flt-1 is expressed on monocytes, renal mesengial cells, Leydig and Sertoli cells (Barleon et al., (1996) Blood. 87:3336-3343; Takahashi et al., (1995) Biochem. Biophys. Res. Commun. 209:218-226; Ergun et al., (1997) Mol. Cell. Endocrinol. 131:9-20). Flk-1 is also expressed on Leydig and Sertoli cells and on hematopoietic stem cells and megakaryocytes (Ergun et al., (1997) Mol. Cell. Endocrinol. 131:9-20; Katoh et al., (1995) Cancer Res. 55:5687-5692; Yang and Cepko (1996) J. Neurosci. 16:6089-6099). Further, VEGF exerts its multiple actions by binding to Flt-1 and Flk-1 and not on Flt-4. Many studies show that Flt-1 and Flk-1 receptors may play a leading role in VEGF induced angiogenesis; however, they seem to be involved in different biological activities.

Antisense compounds are commonly used as research and diagnostic reagents. For example, antisense oligonucleotides, which are able to inhibit gene expression with exquisite specificity, are often used by those of ordinary skill in the relevant art the to elucidate the function of particular genes. Antisense compounds are also used, for example, to distinguish between functions of various members of a biological pathway. Antisense modulation has, therefore, been harnessed for research use. The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

Antisense technology is emerging as an effective means for blocking or inhibiting the expression of specific gene products and, therefore, can be uniquely useful in a number of therapeutic, diagnostic, and research applications involving the modulation of VEGF receptor expression. The effective regulation of pathological angiogenesis using the antisense oligonucleotides of the present invention can be useful in medical treatments for various diseases and disorders including, but not limited to, inflammation, tumour growth and metastasis, ocular diseases, arthritis, psoriasis and atherosclerosis.

This background information is provided for the purpose of making known information believed by the applicant to be of possible relevance to the present invention. No admission is necessarily intended, nor should be construed, that any of the preceding information constitutes prior art against the present invention. Publications referred to throughout the specification are hereby incorporated by reference in their entireties in this application.

### SUMMARY OF THE INVENTION

An object of the present invention is to provide anti-angiogenic antisense oligonucleotides directed toward mammalian VEGF receptors and uses thereof. In accordance with an aspect of the present invention, there is provided an antisense oligonucleotide complementary to a gene encoding a mammalian vascular endothelial growth factor (VEGF) receptor selected from the group comprising Flt-1 and Flk-1, wherein said antisense oligonucleotide comprises about 15 to about 25 nucleotides complementary to said gene.

In accordance with an aspect of the present invention, there is provided an antisense oligonucleotide complementary to a gene encoding a mammalian vascular endothelial growth factor (VEGF) receptor selected from the group comprising Flt-1 and Flk-1, wherein said antisense oligonucleotide comprises about 15 to about 25 nucleotides complementary to said gene and wherein the VEGF receptor is a non-bovine receptor.

In accordance with another aspect of the invention, there is provided a pharmaceutical composition comprising a pharmaceutically acceptable diluent and an antisense oligonucleotide complementary to a gene encoding a mammalian VEGF receptor selected from the group comprising Flt-1 and Flk-1, wherein said antisense oligonucleotide comprises about 15 to about 25 nucleotides complementary to said gene.

In accordance with another aspect of the invention, there is provided a method of blocking pathological angiogenesis in a mammal in need of such therapy, comprising the step of administering to said mammal an antisense oligonucleotide complementary to a gene encoding a mammalian VEGF receptor selected from the group comprising Flt-1 and Flk-1.

In accordance with another aspect of the invention, there is provided a method of blocking inflammation in a mammal in need of such therapy, comprising the step of administering to said mammal the antisense oligonucleotide an antisense oligonucleotide complementary to a gene encoding a mammalian VEGF receptor selected from the group comprising Flt-1 and Flk-1.

### BRIEF DESCRIPTION OF THE FIGURES

FIG. 1: Antisense regulation of VEGF receptors expression on bovine aortic endothelial cells (BAEC). BAEC were seeded at 1×10⁶ cells/100 mm culture plate and grown to confluence. Cells were treated either with antisense or scrambled sequences. Inimunoprecipitation was performed on 12 mg of total proteins as described in Example I. The immunoprecipitated proteins were subjected to SDS-polyacrylamide gel electrophoresis under reducing conditions. Flt-1 and Flk-1 protein expression was revealed by Western blot analysis. Image densitometry results are given as relative expression percentage as compared to PBS-treated cells (control=Ctrl). A) Flt-1 protein expression of PBS-treated cells (Ctrl), cells treated with antisense Flt-1 oligomers (AS1-Flt and AS2-Flt; 10⁻⁷ M), or cells treated with the scrambled Flt-1 oligomer (SCR-Flt; 10⁻⁷ M). B) Flk-1 protein expression of PBS-treated cells (Ctrl), cells treated with antisense Flk-1 oligomers (AS1-Flk and AS2-Flk; 10⁻⁷ M), or cells treated with the scrambled Flk-1 oligomer (SCR-Flk; 10⁻⁷ M). C) Flk-1 protein expression of PBS-treated cells (Ctrl), cells treated with antisense Flk-1 oligomers (AS1-Flk and AS2-Flk; 5×10⁻⁷ M), or cells treated with the scrambled Flk-1 oligomer (SCR-Flk; 5×10⁻⁷ M).

FIG. 2: Western blot analysis of antisense cross-reactivity. BAEC were seeded at 1×10⁶ cells/100 mm culture plate and grown to confluence. Cells were treated either with antisense AS1-Flk or AS2-Flt. Total proteins were isolated and immunoprecipitated against the mentioned receptor. Image densitometry results are given as relative expression (%) as compared to PBS-treated cells (Ctrl). A) Flt-1 protein expression of PBS-treated cells (Ctrl), cells treated with the more potent antisense Flk-1 oligomer (AS1-Flk; 5×10⁻⁷ M), or cells treated with the more potent antisense Flt-1 oligomer (AS2-Flt; 5×10⁻⁷ M). B) Flk-1 protein expression of PBS-treated cells (Ctrl), cells treated with the more potent antisense Flk-1 oligomer (AS1-Flk; 5×10⁻⁷ M), or cells treated with the most potent antisense Flt-1 oligomer (AS2-Flt; 5×10⁻⁷ M).

FIG. 3: Antisense regulation of VEGF-induced Flt-1 and Flk-1 phosphorylation. A) Analysis of Flt-1 phosphorylation of PBS-treated cells (Ctrl), unstimulated (−) or stimulated (+) with VEGF, and from cells treated either with the most potent antisense Flk-1 oligomer (ASI-FPk; 5×10⁻⁷ M) with VEGF stimulation (+), or cells treated with the most potent antisense Flt-1 oligomer (AS2-Flt; 5×10⁻⁷M) with VEGF stimulation (+). B) Analysis of Flk-1 protein phosphorylation of BAEC treated as described for A.

FIG. 4: Mitogenic effect of VEGF and PlGF on endothelial cell proliferation. BAEC were seeded at 1×10⁴ cells/well (24-well tissue culture plate) and stimulated for 24 h with DMEM culture media, 5% FBS. The cells were synchronized in Go by a 48 h treatment with DMEM, 0.25% FBS. The cells were then stinmulated with VEGP (10⁻¹¹, 10⁻¹⁰ and 2.5×10⁻¹⁰ M) or PlGF (10⁻¹⁰, 2.5×10⁻¹⁰, 10⁹ and 10⁻⁸M), and cell number was counted 72 h post-treatment. The values are means of cell count obtained from 6 wells for each treatment. [*, p<0.05; ***, p<0.001 as compared with control (DMEM, 1% FBS) as determined by analysis of variance followed by an unpaired Student's t-test.]

FIG. 5: Effect of antisense oligomers on VEGF-induced endothelial cell proliferation. BAEC were seeded at 1×10⁴ cells/well (24-well tissue culture plate) and stimulated for 24 h with DMEM culture media and 5% BBS with or without antisense oligomers (10⁻⁷ M), the cells were synchronized by a 48 h treatment with DMEM and 0.25% FBS with or without antisense oligomers (10⁻⁷ M daily). The cells were then stimulated with VEGF (10⁻⁹ M) with or without antisense oligomers (10⁻⁷ M daily), and cell number was counted 72 h post-treatment. The values present are means of cell count obtained from 10 wells for each treatment. [**, p<0.01 as compared with control (DMEM, 1% FBS); ††, p<0.01 as compared with VEGF (2.5×10⁻¹⁰ M) as determined by analysis of variance followed by an unpaired Student's t-test.]

FIG. 6: Chemotactic effect of VEGP and PlGF on endothelial cell migration. BAEC were trypsinized and resuspended in DMEM, 1% FBS, and antibiotics; and 5×10⁴ cells were added in the higher chamber of the modified Boyden chamber apparatus, and the lower chamber was filled with DMEM, 1% FBS and antibiotics with or without VEGF or PlGF. Five hours (5 h) post-incubation at 37° C., the migrated cells were stained and counted by using a microscope adapted to a digitized videocamera. The values are means of migrating cells/mm² from 6 chambers for each treatment. [**, p<0.01; ***, p<0.001 as compared with control buffer (PBS) as determined by analysis of variance followed by an unpaired Student's t-test.]

FIG. 7: Antisense oligomer effects on VEGF-induced endothelial cell migration. BAEC were trypsinized and seeded at 2.5×10⁶ cells/well of 6-well tissue culture plate, stimulated for 24 h in DMEM, 5% FBS, and antibiotics with or without antisense oligomers (10⁻⁷ M), starved for 48 h in DMEM, 0.25% FBS, and antibiotics with or without antisense oligomers (10⁻⁷ M daily). Cells were harvested by trypsinization, resuspended in DMEM, 1% FBS, and antibiotics. Cells, 5×10⁴, with or without antisense oligomers (10⁻⁷ M) were added in the higher chamber of the modified Boyden chamber apparatus, and the lower chamber was filled with DMEM, 1% FBS, and antibiotics plus VEGF. Five (5) h post-incubation at 37° C., the migrated cells were stained and counted by using a microscope adapted to a digitized videocamera. The values are means of migrating cells/mm² from 6 chambers for each treatment. [*, p<0.05; **, p<0.01 as compared with control-PBS. \\, p<0.01; †††, p<0.001 as compared with control-VEGF (10⁻⁹ M) as determined by analysis of variance followed by an unpaired Student's t-test.]

FIG. 8: VEGF and placental growth factor (PlGF) effect on endothelial cell platelet activating factor (PAF) synthesis. Confluent BAEC (6-well tissue culture plate) were incubated with ³H-acetate and were stimulated with either VEGF or PlGF for 15 min. The radioactive polar lipids samples were extracted by the Bligh and Dyer procedure (Bligh and Dyer (1959) Can. J. Biochen. Physiol. 37, 911). The samples were injected into a 4.6×250 mm Varian Si-5 column and eluted with a mobile phase (H₂O:CHCl₃:MeOH; 5:40:55; 0.5 ml/min). Fractions were collected every minute after injection, and radioactivity was determined with a β-counter. The values are means of at least eight experiments. [*, p<0.05; ***, p<0.001 as compared with control buffer (PBS) as determined by analysis of variance followed by an unpaired Student's t-test.]

FIG. 9: Effect of antisense oligomers on VEGF-induced PAF synthesis to assess the role of VEGF receptors on PAF synthesis. BAEC were seeded at 2.5×10⁵ cells/well of 6 well tissue culture plate, stimulated for 24 h in DMEM, 5% FBS, and antibiotics with or without antisense oligomers (₁₀⁻⁷−5×10⁻⁷ M) and starved for 48 h in DMEM, 0.25% FBS, and antibiotics with or without antisense oligomers (10⁻⁷−5×10⁻⁷ M daily) for Gₒ synchronization. The cells were then grown to confluence for 24 h in DMEM, 1% FBS, and antibiotics with or without antisense oligomers (10⁻⁷−5×10⁻⁷ M) and starved for 8 h in DMEM, 0.25% FBS, and antibiotics with or without antisense oligomers (10⁻⁷−5×10⁻⁷ M) to induce an upregulation of VEGF receptor expression. Then the cells were incubated with ³H-acetate, and treated with VEGF (10⁻⁹ M). The values are means of at least eight experiments. [*, p<0.05; **, p<0.01; and ***, p<0.001, as compared with control buffer (PBS). †††, p<0.001 as compared with VEGF (10⁻⁹ M) as determined by analysis of variance followed by an unpaired Student's t-test.]

FIG. 10: Assessment of the correlation between antisense Flk-1 oligomer regulation of Flk-1 expression and VEGF-induced PAF synthesis. Shown is the expression of Flk-1 protein expression of BAEC untreated or treated with antisense Flk-1 oligomers (10⁻⁷−5×10⁻⁷ M) versus PAF synthesis elicited by a treatment with VEGF (10⁻⁹ M).

FIG. 11: Surgical procedure. An incision of the skin was made just above the right thigh of the mouse (A), an incision of the rectus sheath was made to access the abdominal cavity, and the right testis was pulled out through the inguinal canal (B), a fine needle 25G5/8 was used to create a small hole at the base of the testis where there are no apparent blood vessels (C), a small catheter (PE10) was inserted in the hole made at the base of the testis (D), the catheter was fixed at the base of the testis to avoid its movement into the testis (E). Pictures of 4 different regions of the testis (A1, A2, B1, B2) at different magnifications were taken with a digital camera (F), the testis was reinserted into the scrotum by passing through the inguinal canal, and the rectus sheath sutured (G), a mini-osmotic pump pre-filled with the substance to be infused into the testis was attached to the free extremity of the catheter. The pump was placed under the skin on the abdominal right flank and the skin was finally sutured (H).

FIG. 12: ITEGF-angiogenic effect and its inhibition by antisen?se oligonucleotide gene therapy: A sustained infusion of control vehicle (PBS) had no or marginal angiogenic effect (A), VEGF-infusion for 14 days induced the formation of new blood vessels (arrows) (B), treatment with antisense oligomer (AS) targeting either Flk-1 (C) or Flt-1 (D) mRNA abrogated VEGF angiogenic activity. (Stereomicroscopic pictures were taken at 48× of magnification).

FIG. 13: VEGF-angiogenic effect and its inhibition by antisense oligonucleotide gene therapy: A: Effect of a sustained infusion of VEGF (1, 2.5 and 5 μg) on a 14 day period on the formation of new blood vessels in mouse testis as compared to control sham operated and PBS treated groups. B: Combination of antisense oligomers targeting either Flk-1 or Flt-1 mRNA (AS-Flk-1 or AS-Flt-1; 200 μg) to VEGF (2.5 μg) abrogated the formation of new blood vessels, whereas, scrambled oligomers (AS-Scr; 200 μg) did not prevent VEGF-angiogenic activity. n=5 to 11 animals per treatment. **P<0.01 and ***P<0.001 vs SHAM; ††P<0.01 vs VEGF.

FIG. 14: VEGF-vasodilatory effect on pre-existing blood vessels and its inhibition by antisense oligonucleotide gene therapy: A: In a sham operated control group, there is no change in the diameter of pre-existing blood vessels at day 14 and 17 post-procedure. VEGF (2.5 μg) infusion on a 14 days period did not modulate the vascular tone of pre-existing blood vessels with a diameter smaller than 20 μm. However, VEGF increased significantly the diameter of pre-existing blood vessels with a diameter from 20 to 100 μm as compared to untreated arteries (day 0). The arrest of VEGF infusion abrogated its vasodilatory effect within 3 days (day 17). B: Combination of antisense oligomers targeting either Flk-1 or Flt-1 mRNA (AS-Flk-1 or AS-Flt-1; 200 μg) to VEGF (2.5 μg) inhibited the vasodilatory effect of VEGF on pre-existing vessels with a diameter from 20 to 100 μm. Addition of a scrambled oligomer to VEGF did not inhibit VEGF vasodilatory effect. n=4 to 11 animals per treatment. ++P<0.01 vs DAY 0;: ‡ P<0.05 vs DAY 14; ** P<0.01 and *** P<0.001 vs SHAM; ††P<0.01 vs VEGF.

FIG. 15: VEGF-angiogenic effect and vasodilatory effect on new blood vessels: Number and diameter (μm) of new blood vessels after 14 days of treatment with VEGF (2.5 μg) and at day. 17 (3 days post-VEGF). In a sham operated group, the number and diameter of new blood vessels remained the same at day 14 and 17 post-procedure. A treatment with VEGF (2.5 μg) for 14 days increased the formation of blood vessels as compared to the control sham operated group, and the diameter of the new blood vessels was not statistically different from those observed in control sham operated mice. Three days after the arrest of VEGF infusion (day 17), the number and the diameter of the new blood vessels remained the same as observed at day 14 under VEGF treatment. n=4 to 11 animals per treatment.; ** P<0.01 and *** P<0.001 vs SHAM.

FIG. 16: Flk-1 protein expression: Positive Flk-1 protein expression on vascular endothelial cells was detected by immunohistochemistry (cells stained in brown; arrow). Basal expression in control sham operated mice (A); VEGF infusion maintained the level of Flk-1 protein expression (B); a treatment with AS-Flk-1 prevented Flk-1 protein expression (C); whereas a treatment with either an AS-Flt-1 (D) or a scrambled oligomer (E) did not alter the vascular Flk-1 protein expression (Magnification 1000×).

FIG. 17: Flt-1 protein expression: Positive Flt-1 protein expression on vascular endothelial cells, was detected by immunohistochemistry (cells stained in brown; arrow). Basal expression in control sham operated mice (A); VEGF infusion maintained the level of Flt-1 protein expression (B); a treatment with AS-Flk-1 did not alter Flt-1 protein expression (C); a treatment with AS-Flt-1 prevented Flt-1 protein expression (D); whereas a scrambled oligomer did not alter the vascular Flt-1 protein expression (E) (Magnification 1000×).

FIG. 18: ecNOS protein expression: Positive ecNOS protein expression on vascular endothelial cells was detected by immunohistochemistry (cells stained in brown; arrow). Basal expression in control sham operated mice (A); VEGF infusion maintained the level of ecNOS protein expression (B); a treatment with either AS-Flk-1 (C), AS-Flt-1 (D) or a scrambled oligomer (E) did not alter the vascular ecNOS protein expression (Magnification 1000×).

FIG. 19: Effects of intraocular injections of antisense oligonucleotides complementary to VEGF receptors on neovascular buds density in a mouse model of retinopathy.

FIG. 20: Effects of intraocular injections of antisense oligonucleotides complementary to VEGF receptors on retinal microvessels density in a mouse model of retinopathy.

### DETAILED DESCRIPTION OF THE INVENTION

The present invention employs antisense oligonucleotides for use in modulating the function of nucleic acid molecules encoding vascular endothelial growth factor (VEGF) receptors Flt-1 and Flk-1, ultimately modulating the amount of VEGF receptor protein produced. This is accomplished by providing antisense compounds which specifically hybridise with one or more nucleic acids encoding vascular endothelial growth factor (VEGF) receptors Flt-1 and Flk-1. The specific hybridisation of an oligonucleotide with its target nucleic acid interferes with the normal function of the nucleic acid. This modulation of function of a target nucleic acid by compounds which specifically hybridise to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of vascular endothelial growth factor (VEGF) receptors, Flt-1 and/or Flk-1.

Definitions

Unless defined otherwise, all technical and scientific terms used herein have the same meaning as commonly understood by one of ordinary skill in the art to which this invention belongs.

“Antisense oligonucleotide”, as used herein, refers to any oligonucleotide that is complementary to the target gene. The antisense oligonucleotide may be in the form of DNA, RNA or any combination thereof.

“Corresponds to” refers to a polynucleotide sequence is homologous (i.e., is identical, not strictly evolutionarily related) to all or a portion of a reference polynucleotide sequence, or that a polypeptide sequence is identical to a reference polypeptide sequence.

“Naturally-occurring”, as used herein, as applied to an object, refers to the fact that an object can be found in nature. For example, a polypeptide or polynucleotide sequence that is present in an organism (including viruses) that can be isolated from a source in nature and which has not been intentionally modified in the laboratory is naturally-occurring.

“Nucleic acid” refers to DNA and RNA and can be either double stranded or single stranded. The invention also includes nucleic acid sequences which are complementary to the claimed nucleic acid sequences.

“Oligonucleotide”, as used herein, refers to an oligomer or polymer of ribonucleic acid (RNA) or deoxyribonucleic acid (DNA) or mirnetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

“Polynucleotide” refers to a polymeric form of nucleotides of at least 10 bases in length, either ribonucleotides or deoxynucleotides or a modified form of either type of nucleotide. The term includes single and double stranded forms of DNA or RNA.

“Protein”, as used herein, refers to a whole protein, or fragment thereof, such as a protein domain or a binding site for a second messenger, co-factor, ion, etc. It can be a peptide or an amino acid sequence that functions as a signal for another protein in the system, such as a proteolytic cleavage site.

Other biochemistry and chemistry terms herein are used according to conventional usage in the art, as exemplified by The McGraw-Hill Dictionary of Chemical Terms (ed. Parker, S., 1985), McGraw-Hill, San Francisco).

In one embodiment of the present invention antisense oligonucleotides are designed that are complementary to specific regions of mammalian Flt-1 and Flk-1 genes. In a specific embodiment of the present invention antisense oligonucleotides are designed that are complementary to specific regions of the human Flt-1 and Flk-1 genes.

Exemplary antisense oligonucleotide sequences of the present invention are listed below. It should be apparent to one skilled in the art that other antisense oligonucleotide sequences that are complementary to specific regions of mammalian Flt-1 and Flk-1 genes are within the scope of the present invention.

| | |
|---|---|
| BOVINE FLT-1 | |
| AS1-bFlt-1: | |
| 5′-CAA AGA TGG ACT CGG GAG-3′ | (SEQ ID NO:1) |
| AS2-bFlt-1: | |
| 5′-GTC GCT CTT GGT GCT ATA-3′ | (SEQ ID NO:2) |
| BOVINE FLK-1 | |
| AS1-bFlk-1: | |
| 5′-GCT GCT CTG ATT GTT GGG-3′ | (SEQ ID NO:3) |
| AS2-bFlk-1: | |
| 5′-CCT CCA CTC TTT TCT CAG-3′ | (SEQ ID NO:4) |
| MURINE FLT-1 | |
| AS1-mFlt-1: | |
| 5′-AAG CAG ACA CCC GAG CAG-3′ | (SEQ ID NO:5) |
| AS2-mFlt-1: | |
| 5′-CCC TGA GCC ATA TCC TGT-3′ | (SEQ ID NO:6) |
| MURINE FLK-1 | |
| AS1-mFlk-1: | |
| 5′-AGA ACC ACA GAG CGA CAG-3′ | (SEQ ID NO:7) |
| AS2-mFlk-1: | |
| 5′-AGT ATG TCT TTC TGT GTG-3′ | (SEQ ID NO:8) |
| HUMAN FLT-1 | |
| AS1-hFlt-1: | |
| 5′-CTG TTT CCT TCT TCT TTG-3′ | (SEQ ID NO:9) |
| AS2-hFlt-1: | |
| 5′-TCC TTA CTC ACC ATT TCA-3′ | (SEQ ID NO:10) |
| AS3-hFlt-1: | |
| 5′-TGT TTC CTT CTT CTT TGA-3′ | (SEQ ID NO:11) |
| AS4-hFlt-1: | |
| 5′-TAC TCA CCA TTT CAG GCA-3′ | (SEQ ID NO:12) |
| AS5-hFlt-1: | |
| 5′-ACT CAC CAT TTC AGG CAA-3′ | (SEQ ID NO:13) |
| HUMAN FLK-1/KDR | |
| AS1-hFlk-1: | |
| 5′-AGT ATG TCT TTT TGT ATG-3′ | (SEQ ID NO:14) |
| AS2-hFlk-1: | |
| 5′-TGA AGA GTT GTA TTA GCC-3′ | (SEQ ID NO:15) |
| AS3-hFlk-1: | |
| 5′-ACT GCC ACT CTG ATT ATT-3′ | (SEQ ID NO:16) |
| AS4-hFlk-1: | |
| 5′-TTT GCT CAC TGC CAC TCT-3′ | (SEQ ID NO:17) |
| AS5-hFlk-1: | |
| 5′-GTC TTT TTG TAT GCT GAG-3′ | (SEQ ID NO:18) |

Design and Preparation of Antisense Oligonucleotides

“Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a mammalian VEGF receptor that is Flt-1 or Flk-1. As used herein the “gene encoding a VEGF receptor” refers to any gene which encodes a protein that is capable of acting as a VEGF receptor. Such gene sequences can be available on electronic databases, for example, GenBank. In the present invention the bovine antisense oligonucleotides were designed from the sequence in GenBank Accession Nos. X94263 and X94298, the murine antisense oligonucleotides were designed from the sequence in GenBank Accession Nos. D28498 and X70842, and the human antisense oligonucleotides were designed from the sequence in GenBank Accession Nos AF063658 and X51602. It would be readily appreciated by a worker skilled in the art that further mammalian Flt-1 and Flk-1 gene sequences can be obtained using the publicly available databases and that the accession numbers provided herein do not limit the scope of the present invention.

The targeting process also includes determination of a site or sites within this gene for the antisense interaction to occur such that the desired effect, e.g., detection or modulation of expression of the protein, will result. Within the context of the present invention, a possible intragenic site is the region encompassing the translation initiation or termination codon of the open reading frame (ORF) of the gene. It is known in the art that eukaryotic and prokaryotic genes may have two or more alternative start codons, any one of which may be utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding a mammalian VEGF receptor that is Flt-1 or Flk-1, regardless of the sequence(s) of such codons.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N⁷-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also potential targets. It has also been found that introns can be effective target regions for antisense compounds targeted, for example, to DNA or pre-mRNA.

Once one or more target sites have been identified, oligonucleotides are chosen which are sufficiently complementary to the target, i.e., hybridise sufficiently well and with sufficient specificity, to give the desired effect.

In the context of this invention, “hybridisation” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridisable. An antisense compound is specifically hybridisable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Selection of the Antisense Structure

RNA vs DNA: Native mRNA can be hybridised with complementary (antisense) DNA or RNA fragments. Single-stranded oligoribonucleotides are extremely sensitive to ribonucleases, whereas oligodeoxyribonucleotides (ODN) are less sensitive, and may be used for transient application.

Size and Chemical modification: The ODN contains 12-15 bases to recognise a single genornic sequence. Fortuitously, the oligomer length required to hybridise effectively with its complement is also approximately the same size. Natural ODNs (≈4-8 kDa) are negatively charged and their cellular endocytosis is mediated by two surface proteins of 34 and 80 kDa. The ODN hybridises with its complementary mRNA sequence, prevents mRNA processing and translation into protein. Another advantage of using DNA, rather than RNA oligomers, is the specific recognition of the DNA oligomer-mRNA hybrid by the nuclease RNase H. This enzyme may cleave the RNA at the duplex site and reduce in part the mRNA concentration available for translation. Yet, even the more stable ODN has a half life (<2-3 hrs) too short to be clinically effective.

Chemical modifications of the ODNs can improve the stability and the intracellular incorporation. One embodiment of the present invention provides antisense oligonucleotides that have been modified by replacement of the negatively charged oxygen on the internucleotide phosphate bridge by a sulphur atom, which, increases nuclease resistance, maintains hybridisation capacity, stimulates RNase H activity and does not add toxicity.

Targeted gene and targeted mRNA region: The gene targeted by the antisense is also critical, and requires special consideration: 1) the targeted protein should play a unique biological role with “no substitute protein” capable of carrying out similar function in the cell. Any segment of the mRNA can be targeted with antisense-oligodeoxyribonucleoide (AS-ODN) sequences, however, empirical data has demonstrated that ODNs directed near the AUG initiation site were most effective at inhibiting gene expression.

Antisense gene sequence: Recent reports suggested that the presence of 4 contiguous guanosines (GGGG) within the sequence of a phosphorothioate oligodeoxynucleotides might induce non-specific effects. Oligodeoxynucleotides are polyanions capable of binding to heparin-binding proteins such as aFGF, bFGF, PDGF and VEGF, this effect is heavily dependent on the presence of GGGG in the oligomer and should be avoided for future investigations.

The antisense oligonucleotides of the present invention range in length from 7 to 50 nucleotides.

In one embodiment of the present invention the antisense oligonucleotides are selected to have the following characteristics:

i) no more than three, or preferably less, consecutive guanosines;

ii) incapacity to form hairpin structures;

iii) minimal capacity to form homodimers; and

iv) contain between about 15 and about 25 nucleotides that are complementary to the target gene.

In a related embodiment the of the present invention the antisense oligonucleotides are selected to have the above characteristics i) to iii) and contain between 15 and 20 nucleotides. In an alternative embodiment of the present invention the antisense oligonucleotides contain 18 nucleotides.

The antisense oligonucleotides can be selected, based on the above characteristics, using commercially available computer software, for example OLIGO® Primer Analysis.

While antisense oligonucleotides are one form of antisense compounds, the present invention contemplates other oligomeric antisense compounds, including but not limited to oligonucleotide mimetics such as are described below. As is known in the art, a nucleoside is a base-sugar combination. The base portion of the nucleoside is normally a heterocyclic base. The two most common classes of such heterocyclic bases are the purines and the pyrimridines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Alternative modified oligonucleotide backbones include, for example, pbosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Alternative modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In alternative oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al (1991) Science, 254, 1497−1500.

Modified oligonucleotides may also contain one or more substituted sugar moieties. For example, oligonucleotides may comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C, to C₁₀ alkyl or C₂ to C₁₀ alkenyl and alkynyl. Particularly preferred are O[(CH₂)ₙO]ₘ CH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

Other modifications include 2′-methoxy (2′-O—CH₃), 2′-aminopropoxy (2′-OCH₂ CH₂ CH₂ NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschw;itz, J. I., ed. John Wiley & Sons, 1990, those disclosed by Englisch et al (1991) Angewandte Chemie, International Edition, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278), even more particularly when combined with 2′-O-methoxyethyl sugar modifications.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et at (1989) Proc. Natl. Acad. Sci. USA, 86, 6553-6556), cholic acid (Manoharan et al (1994) Bioorg. Med. Chem. Lett., 4, 1053-1060), a thioether, e.g., hexyl-S-tritylthiol (Manolharan et al (1992) Ann. N.Y. Acad. Sci., 660, 306-309; Manoharan et al (1993) Bioorg. Med. Chem. Lett., 3, 2765-2770), a thiocholesterol (Oberhauser et al (1992) Nucl. Acids Res., 20, 533-538), an aliphatic chain, e.g., dodecandiol or undecyl residues (Saison-Behmoaras et al (1991) EMBO J., 10, 1111-1118), a phospholipid, e.g., di-hexadecyl-rac-glycerol or triethylammonium 1,2-di-O-hexadecyl-rac-glycero-3-H-phosphonate (Manoharan et al (1995) Tetrahedron Lett., 36, 3651-3654; Shea et al (1990) Nucl. Acids Res., 18, 3777−3783), a polyamine or a polyethylene glycol chain (Manoharan et al (1995) Nucleosides & Nucleotides, 14, 969-973), or adamantane acetic acid (Manoharan et al (1995) Tetrahedron Lett., 36, 3651-3654), a palmityl moiety (Mishra et al (1995) Biochim. Biophys. Acta, 1264, 229-237), or an octadecylamine or hexylamino-carbonyl-oxycholesterol moiety (Crooke et al (1996) J. Pharmacol. Exp. Ther., 277, 923-937.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides may contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

An expression vector comprising the antisense oligonucleotide sequence may be constructed having regard to the sequence of the oligonucleotide and using procedures known in the art.

Vectors can be constructed by those skilled in the art to contain all the expression elements required to achieve the desired transcription of the antisense oligonucleotide sequences. Therefore, the invention provides vectors comprising a transcription control sequence operatively linked to a sequence which encodes an antisense oligonucleotide. Suitable transcription and translation elements may be derived from a variety of sources, including bacterial, fungal, viral, mammalian or insect genes. Selection of appropriate elements is dependent on the host cell chosen.

Testing Activity of Antisense Oligonucleotides

One embodiment of the present invention provides methods for testing the activity of the antisense oligonucleotides. Commonly the antisense oligonucleotides are first tested in vitro to determine modulation of VEGF receptor expression and the subsequent effect of this modulation. The oligonucleotides can then be tested using in vivo techniques, using animal models, prior to their testing and subsequent use in humans.

In testing candidate antisense oligonucleotides, the biological end point should always include a demonstration of diminution in concentration of the protein product of the targeted mRNA. To confirm the capacity of the antisense sequence to inhibit the expression of the targeted protein, it is preferable to look at the final product (the protein itself) by immunohistochemistry rather than looking at the mRNA level by in situ hybridisation. The reason is that the antisense recognises the mRNA sequence, binds to it, and prevents its translation into protein. Despite that a fraction of the mRNA may be degraded by the RNase H activity at the hybrid site, it remains that a good fraction will not be degraded by the RNase H activity and will be recognised by in situ hybridisation despite the fact that this mRNA will not necessarily be translated.

Antisense oligomers should not affect protein expression of non-targeted mRNA. To confirm the selectivity of the antisense sequence, it is important to demonstrate that the candidate antisense will not effect the expression of a non-targeted protein which has the closest gene homology with the targeted protein.

In Vitro Assays

The in vitro assays can be performed using cultures of any cell line that expresses the Flt-1 and/or the Flk-1 VEGF receptors. For example, bovine aortic endothelial cells (BAEC) can be used to test bovine antisense oligonucleotides of the present invention and human umbilical vein endothelial cells (HUVEC) can be used to test human antisense oligonucleotides of the present invention.

The BAEC are prepared and tested using techniques known to a worker skilled in the art and as described in Example I of the present application. HUVEC can also be prepared using standard techniques known to a worker skilled in the art, including, but not limited to the technique outline below.

Endothelial Cell Isolation from Human Umbilical Cords

Fresh umbilical cords are put in phosphate buffered saline (PBS) plus antibiotics solution, and can be kept at least for 24 hrs at 4° C. The extremities of the cords are cut; blunted needles are inserted in the major umbilical vein and adapted to stopcocks. A surgical suture is made around the umbilical cords at the level of the needles. The umbilical cords are rinsed with PBS to remove blood borne elements in the veins. A collagenase solution (1 mg collagenase/ml of PBS) is infused in the veins and kept in for 8 minutes at 37° C. Then, the collagenase solution is collected and neutralised with 10% FBS-DMEM solution. Additional 10% FBS-DMEM solution is infused in the veins, and passed back and forth to detach and isolate venous endothelial cells from the cords and added to the previous eluate. The solution is then centrifuged at 1300 rpm, 2 min at room temperature. The supernatant is discarded and the pellet (containing the endothelial cells) resuspended in culture media. The endothelial cells are later characterised by their cobblestone monolayer morphology, Factor VIII immunoprecipitation and by diiodoindocarbo cyanide acetylated LDL uptake, Cells are not passaged for more than 4 cycles to avoid the possibility that repeated trypsinisation might affect receptor expression.

Various assays can be performed using these cell cultures including those used to determine protein expression from the target Flt-1 and/or Flk-1 genes and the downstream effects of decreased protein expression.

Western blot and/or immunohistochemical analysis of Flt-1 and Flk-1 protein expression can be carried out using standard techniques and antibodies specific for Flt-1 or Flk-1. A decrease in protein expression, following treatment of cells in culture with the candidate antisense oligonucleotide, in comparison to untreated cells, is indicative of an effective antisense oligonucleotide. This is demonstrated in Example I. Western blot and/or immunohistochemical analysis can also be used to determine the degree of VEGF-induced Flt-1 and/or Flk-1 phosphorylation. An effective antisense oligonucleotide will cause a decrease in phosphorylation, as demonstrated in Example I.

Mitogenic assays can be performed to monitor endothelial cell proliferation in the presence and absence of a candidate antisense oligonucleotide. Effective antisense oligonucleotides of the present invention (i.e. those that are capable of down-regulating Flt-1 and/or Flk-1 protein expression) can block or inhibit the mitogenic effect of VEGF and thereby reduce endothelial cell proliferation. These assays can be performed using standard techniques well known to those skilled in the art. One example of a mitogenic assay using BAEC cultures is provided in Example I. As indicated above, this assay can be adapted for use with any Flt-1 and/or Flk-1 expressing cell lines.

Chemotactic assays can be performed to evaluate the effect of candidate antisense oligonucleotides on VEGF-mediated cell migration. Effective antisense oligonucleotides of the present invention (i.e. those that are capable of down-regulating Flt-1 and/or Flk-1 protein expression) can block or inhibit the chemotactic effect of VEGF. These assays can be performed using standard techniques well known to those skilled in the art. One example of a chemotactic assay using BAEC cultures is provided in Example I. As indicated above, this assay can be adapted for use with any Flt-1 and/or Flk-1 expressing cell lines.

VEGF, as a result of interaction with VEGF receptors, has been shown to enhance vascular permeability through platelet activating factor (PAF) synthesis. A reduction in PAF synthesis, therefore, can be indicative of a successful antisense effect. By monitoring the amount of PAF produced in response to VEGF, in the presence and absence of a candidate antisense oligonucleotide of the present invention, it is possible to identify a reduction in VEGF activity. Methods of monitoring PAF production are well known to those skilled in the art. One example of a PAF production assay using BAEC cultures is provided in Example I. As indicated above, this assay can be adapted for use with any Flt-1 and/or FPk-1 expressing cell lines.

In Vivo

Once a candidate antisense oligonucleotide is demonstrated to have an effective ill vitro effect, it can then be tested in vivo. These assays are generally performed using animal models, for example the mouse testes model presented in Example II. In general, in vivo assays involve the administration or introduction of a candidate antisense oligonucleotide to a subject and monitoring its effect on Flt-1 and Flk-1 protein production and phosphorylation and angiogensis. Protein production and phosphorylation can be assayed by standard techniques, including Western blot and/or immunohistochemical analysis of tissue extracts. Successful candidate antisense oligonucleotides will demonstrate reduced Flt-1 and Flk-1 protein production and phosphorylation and a reduction in VEGF-mediated angiogenesis and/or inflammation.

Histological and microscopic analysis can also be used, according to standard techniques known in the art, to view formation of blood vessels as an indication of angiogensis. Successful candidate antisense oligonucleotides will demonstrate reduced angiogenesis and, therefore, a reduction in the formation of blood vessels in comparison to tissue from untreated subjects.

One, non-limiting, example of an in vitro animal model has been developed using mice testes. Briefly, the model is created using the following steps:

(i) the inguinal canal is cut open to isolate the right testis;

(ii) a PE-10 catheter is inserted through the tunicae vaginalis and positioned in the right testis;

(iii) the catheter is secured with a microsuture (8.0 silk) outside the testis;

(iv) the abdominal rectus aponevrosis is sutured to recreate the inguinal canal; and

(v) the other extremity of the PE-10 catheter is adapted to an Alzet pump loaded with buffer, a candidate antisense oligonucleotide or combination of antisense oligonucleotides and placed subcutaneously on the abdominal-lateral side.

A second animal model that can be used to test the antisense oligonucleotides of the present invention is based on hyperoxia-induced retinopathy. This model is created using new born mouse pups that are exposed to hyperoxia in the perinatal period (Robinson G. S., et al, (1996) Proc. Natl Acad. Sci. USA. 93: 4851-4856; Hardy P., et al, (1998) Invest. Ophtalmol. Vis. Sci. 39: 1888-1898; Lachapelle P., et al, (1999) Can. J. Physiol. Pharmacol. 77: 48-55; and Nandgaonkar B. N., et al, (1996) Ped. Res. 46: 184-188). The antisense oligonucleotide, a combination of antisense oligonucleotides or buffer alone is applied intraocularly and the degree of retinal neovascularization and budding of neovessels is determined. Measurement of peripheral avascular areas is determined by highlighting vasculature by binary transformation of tonality (Adobe Photoshop) and tracing of the areas processed by digital imaging (NIH 1.6) (Zhang S., (2000) Investigative Ophtalmo. Visual Sci. 41: 887−891). The extend of neovascularization in the treated and control eyes will be determined by counting neovascular cell nuclei extending through the internal limiting membrane into the vitreous. The length and diameter of the new blood vessels will be quantified (Hardy P., et at, (1998) Invest. Ophtalnio. Vis. Sci. 39: 1888-1898; Lachapelle P., et at, (1999) Can. J. Physiol. Pharmacol. 77: 48-55; and Nandgaonkar B. N., et al, (1996) Ped. Res. 46: 184-188). Finally, the expression level of VEGF receptors (Flt-1 and Flk-1), and PCNA will be confirmed by immunohistochemistry.

Use of Antisense Oligonucleotides

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

In one embodiment of the present invention the antisense oligonucleotides are used to block VEGF-mediated effects in a mammal suffering from pathological angiogensis. Pathological angiogenesis is present in tumour growth and metastasis, ocular diseases (diabetic and perinatal hyperoxic retinopathies, age-related macular degeneration), arthritis, psoriasis and atherosclerosis. In a related embodiment of the present invention the antisense oligonucleotides are used to inhibit pathological angiogenesis in a mammal in need of such therapy.

In an alternative embodiment of the present invention the antisense oligonucleotides are used to reduce PAF synthesis and inflammation in a mammal in need of such therapy.

The antisense compounds of the present invention are also useful for research and diagnostics, because these compounds hybridize to nucleic acids encoding a mammalian VEGF receptor that is Flt-1 or Flk-1, enabling sandwich and other assays to easily be constructed to exploit this fact. Hybridization of the antisense oligonucleotides of the invention with a nucleic acid encoding a mammalian VEGF receptor that is Flt-1 or Flk-1 can be detected by means known in the art. Such means may include linkage of a fluorophore to the oligonucleotide, attachment of a reporter gene to the oligonucleotide, conjugation of an enzyme to the oligonucleotide, radiolabelling of the oligonucleotide or any other suitable detection means. Kits using such detection means for detecting the level of a mammalian VEGF receptor that is Flt-1 or Flk-1 in a sample may also be prepared.

Antisense Oligonueleotide Administration

In the context of this invention, to “contact” tissues or cells with an oligonucleotide or oligonucleotides means to add the oligonucleotide(s), usually in a liquid carrier, to a cell suspension or tissue sample, either in vitro or ex vivo, or to administer the oligonucleotide(s) to cells or tissues within an animal, including a human. In one embodiment of the present invention the antisense oligonucleotide(s) is contacted with cells or tissue in vivo or ex vivo and subsequently administered to an animal, including a human. When employed as pharmaceuticals, the antisense oligonucleotides are usually administered in the form of pharmaceutical compositions. The pharmaceutical compositions are prepared by adding an effective amount of an antisense oligonucleotide to a suitable pharmaceutically acceptable diluent or carrier. As such, one embodiment of the present invention provides pharmaceutical compositions and formulations which include the antisense oligonucleotides of the invention.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Methods of delivery of foreign nucleic acids, such as antisense oligonucleotides, are known in the art, such as containing the nucleic acid in a liposome and infusing the preparation into an artery (LeClerc G. et al., (1992) J. Clin Invest. 90: 936-44), transthoracic injection (Gal, D. et al., (1993) Lab Invest. 68: 18-25.). Other methods of delivery may include coating a balloon catheter with polymers impregnated with the foreign DNA and inflating the balloon in the region of arteriosclerosis, thus combining balloon angioplasty and gene therapy (Nabel, E. G. et al., (1994) Hum Gene Ther. 5:1089-94.)

Another method of delivery involves “shotgun” delivery of the naked antisense oligonucleotides across the dermal layer. The delivery of “naked” antisense oligonucleotides is well known in the art. See, for example, Felgner et al., U.S. Pat. No. 5,580,859. It is contemplated that the antisense oligonucleotides may be packaged in a lipid vesicle before “shotgun” delivery of the antisense oligonucleotide.

Another method of delivery involves the use of electroporation to facilitate entry of the nucleic acid into the cells of the mammal. This method can be useful for targeting the antisense oligonucleotides to the cells to be treated, for example, a tumour, since the electroporation would be performed at selected treatment areas.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates

In one embodiment of the present invention the antisense oligonucleotides or the pharmaceutical compositions comprising the antisense oligonucleotides may be packaged into convenient kits providing the necessary materials packaged into suitable containers.

To gain a better understanding of the invention described herein, the following examples are set forth. It should be understood that these examples are for illustrative purposes only. Therefore, they should not limit the scope of this invention in any way.

### EXAMPLES

### Example 1

### VEGF Effect on Endothelial Cell Proliferation, Migration and PAF Synthesis

The methods described herein can be carried out using endothelial cells that express Flt-1 and Flk-1 receptors. Exemplary cells that can be used as described herein, are human umbilical vein endothelial cells (HUVEC) and bovine aortic endothelial cells (BAEC).

To discriminate the contribution of Flt-1 and Flk-1 receptors upon endothelial cell (EC) stimulation by VEGF, selective antisense deoxyribophosphorothioate oligomers, which hybridized specifically with a complementary mRNA sequence and prevented the translation of the targeted mRNA into its protein (Crooke R., (1991) Anticancer Drug Des. 6, 609-646; Loke, S. et al (1989) Proc. Natl. Acad. Sci. U.S.A. 86, 3474-3478; Yakubov, L. A. et al (1989) Proc. Natl. Acad. Sci. U.S.A 86,6454-6458), were used. This antisense gene expression knockdown approach resulted in downregulation of the protein expression of Flt-1 or Flk-1 in a highly selective fashion and thus to evaluate their contribution to the biological activities mediated by VEGF.

The mitogenic, chemotactic and PAF synthesis activities of VEGF on BAEC were studied. Furthermore, the ability of antisense oligonucleotide sequences complementary to Flt-1 or Flk-1 mRNA to modulate VEGF-mediated effects is demonstrated. The activation of Flk-1 was found to be sufficient to mediate the VEGF actions on EC in vitro.

Materials and Methods

Cell Culture: BAEC expressing both VEGF receptors (Barleon, B. et al (1994) J. Cell. Biochem. 54, 56-66) were isolated from freshly harvested aorta, cultured in Dulbecco's modified eagle medium (DMEM; Life Technologies, Burlington, ON) containing 5% fetal bovine serum (Hyclone Lab., Logan Utah), and antibiotics (Sigma Chem., St-Louis, Mo.). BAEC were characterized by their cobblestone monolayer morphology and Factor VIII immunohistochemistry, and were not passaged for more than 9 cycles.

Antisense Oligonucleotide Therapy: To discriminate the contribution of Flt-1 and Flk-1 upon stimulation of EC by VEGF, BAEC were treated with antisense oligonucleotide sequences complementary to bovine Flt-1 or Flk-1 mRNA (GenBank Accession Numbers X94263 and 94298). A total of four different antisense oligonucleotide phosphorothioate backbone sequences were used, two targeting bovine Flt-1 mRNA (antisense 1, AS1-bFlt: 5′-CAA AGA TGG ACT CGG GAG-3′ (SEQ ID NO:1); antisense 2, AS2-bFlt: 5′-GTC GCT CTT GGT GCT ATA-3′ (SEQ ID NO:2)), and two targeting bovine Flk-1 mRNA (antisense 1, AS1-bFlk: 5′-GCT GCT CTG ATT GTT GGG-3′ (SEQ ID NO:3); antisense 2, AS2-bFlk: 5′-CCT CCA CTC TTT TCT CAG-3′ (SEQ ID NO:4)). Two scrambled phosphorothioate sequences (scrambled Flt, SCR-Flt: 5′-AGC TAG GCA CGA GAG TGA-3′ (SEQ ID NO:19); scrambled Flk, SCR-Flk: 5′-TGC TGG CAT GTG CGT TGT-3′ (SEQ ID NO:20)) were also used as negative controls. These sequences were designed with no more than three consecutive guanosines and by minimizing their capacity to form hairpins and homodimers. All sequences were synthesized at the Armand Frappier Institute (Laval, Canada). After synthesis, the oligonucleotides were dried, resuspended in sterile water and quantified by spectrophotometry. The antisense oligomer solutions were by-products-free, as confirmed using denaturing polyacrylamide gel electrophoresis (20%; 7 M urea), based on the known length of the oligonucleotide.

Western blot analysis of Flt-1 and Flk-1 protein expression: The efficiency and specificity of the antisense sequences to block the targeted protein expression were evaluated by Western blot analysis. Confluent BAEC (100 mm tissue culture plate) were washed with DMFM and trypsinized (trypsin-EDTA; Life Technologies). Cells were resuspended in DMEM containing 5% of fetal bovine serum and antibiotics, and a cell count was obtained with a Coulter counter Z1 (Coulter Electronics, Luton, UK). Cells were seeded at 1×10⁶ cells/100 mm tissue culture plate (Becton-Dickinson, Rutherford, N.J.), stimulated for 24 h in DMEM/5% FBS/antibiotics±antisense oligonucleotides (10⁻⁷−5×10⁻⁷ M) and starved for 48 h in DMEM/0.25% FBS/antibiotics±antisense oligonucleotides (10⁻⁷ M daily) for G₀ synchronization. The cells were then grown to confluence for 16 h in DMEM/1% FBS/antibiotics±antisense oligonucleotides (10⁻⁷−5×10⁻⁷ M) and starved for 8 h in DMEM/0.25% FBS/antibiotics i antisense oligonucleotides (10⁻⁷−5×10⁻⁷ M) to induce an upregulation of the VEGF receptors expression. The culture medium was removed and cells were rinsed twice with ice-cold DMEM. Total proteins were prepared by the addition of 500 μl of lysis buffer containing phenylmethylsulfonyl fluoride 1 mM (Sigma), leupeptin 10 μg/ml (Sigma), aprotinin 30 μg/ml (Sigma) and NaVO₃ 1 mM (Sigma). Plates were incubated at 4° C. for 30 min, scraped and the protein concentration was determined with a Bio-Rad protein assay kit (Bio-Rad, Hercules, Calif.). Immunoprecipitation was performed on 12 mg of total proteins for each sample by incubation with rabbit anti-mouse Flk-1 IgG or rabbit anti-human Flt-1 IgG polyclonal antibodies (Santa Cruz Biotech., Santa Cruz, Calif.) bound to protein A-Sepharose beads at 4° C. for 1 h. Both antibodies were specific for their targeted protein and do not cross react with each other. After washing 3 times with lysis buffer, the immunoprecipitates were dissolved in Laemmli's buffer, boiled for 5 min in reducing conditions, separated by a 10%-20% gradient SDS-PAGE (Protean II kit; Bio-Rad) and transblotted onto a 0.45-μm polyvinylidene difluoride membranes (Milipore Corp., Bedford, Mass.). The membranes were blocked in 5% Blotto-TTBS (5% nonfat dry milk, Bio-Rad; Tween-20 0.05%, 0.15M NaCl, 25 mM Tris-HCl pH 7.5) for 2 h at room temperature with gentle agitation and incubated for 45 min in 1%. Blotto-TTBS containing the desired antisera (anti-Flt-1 or anti-Flk-1; dilution 1:100). Membranes were washed 3 times with TTBS, reblocked for 10 min in 1% Blotto-TTBS and incubated with a horseradish peroxidase goat anti-rabbit IgG antibodies (dilution 1:7500, Santa Cruz) in 5% Blotto-TTBS for 30 min. Membranes were washed with TTBS, and horseradish peroxidase bound to secondary antibody was revealed by chemiluminescence (Renaissance kit, New England Nuclear, Boston, Mass.). Kaleidoscope molecular weight and SDS-PAGE broad range marker proteins (Bio-Rad) were used as standards for SDS-PAGE. Digital image densitometry (PDI Bioscience, NY) was performed on X-ray films to determine relative percentages of Flt-1 or Flk-1 protein expression.

Western blot analysis of Flt-1 and Flk-1 protein phosphorylation: BAEC were pretreated with the antisense sequences as described above for Western blot analysis. Cells were then rinsed with DMEM, incubated on ice in DMEM+1 mg/ml BSA+VEGF (10⁻⁹ M) for 30 min, incubated at 37° C. for 7 min and then brought back on ice. Cells were rinsed with DMEM+NaVO₃ (1 mM), and total proteins were prepared as described. Inmmunoprecipitation was performed on 500 μg of total proteins with rabbit anti-mouse Flk-1 IgG or rabbit anti-human Flt-1 IgG polyclonal antibodies (Santa Cruz Biotech.) bound to protein G-Sepharose 4 Fast Flow (Amersham, Uppsala, Sweden) at 4 IC for 1 h. After 3 washes with lysis buffer, the immunoprecipitates were dissolved in Laemmli's buffer, boiled for 5 min in reducing conditions, separated by a 6% SDS-PAGE (Mini-Protean II kit; Bio-Rad) and transblotted onto a 0.45 μm PVDF membrane. The membranes were blocked in 3%-BSA-PBST (Tween 0.1 Olo) for 1 h at room temperature and incubated overnight with the primary antisera (mouse anti-phosphotyrosine clone 4G10; dilution 1:3000, Upstate Biotechnology Inc, Lake Placid, N.Y.). Membranes were washed with PBST, incubated with an anti-mouse IgG (dilution 1:4000, Santa Cruz), washed with PBST and chemiluminescence protocol was followed as described above.

Mitogenic assays: Confluent BAEC were washed with DMEM, and trypsinized. Cells were resuspended in 9 ml of DMEM/5% FBS/antibiotics, and a cell count was obtained. BAEC were seeded at 1×10⁴ cells/well of 24-well tissue culture plates, stimulated for 24 h in DMEM 15% FBS/antibiotics±antisenses (10⁻⁷ M) and starved for 48 h in DMEM/0.25% FBS/antibiotics±antisenses (10⁻⁷ M daily) for G₀ synchronization. The cells were stimulated for 72 h in DMEM/1% FBS/antibiotics±antisenses (10⁻⁷ M daily) with different concentrations of VEGF or PlGF (human recombinant vascular growth factor, VEGF₁₆₅; PeproTech Inc., Rocky Hill, N.J., and human placenta growth factor, PlGF₁₅₂; R & D Systems, Minneapolis, Minn.). The cells were then trypsinized and cell number was determined by using a Coulter counter.

Chemotaxis assays: Cell migration was evaluated using a modified Boyden 48-well microchamber kit (NeuroProbe, Cabin John, Md.). Near confluent BAEC (100 mm tissue culture plate) were washed with DMEM, and trypsinized. Cells were resuspended in DMEM/5% FBS/antibiotics, and a cell count was obtained. BAEC were seeded at 2.5×10⁵ cells/well of 6-well tissue culture plates, stimulated for 24 h in DMEM/5% FBS/antibiotics±antisense oligonucleoitdes (10⁻⁷ M), starved for 48 h in DMEM/0.25% FBS/antibiotics±antisense oligonucleotides (10-7 M daily). Cells were harvested by trypsinisation, resuspended in DMEM/1% FBS/antibiotics at a concentration of 1×10⁶ cells/ml. Fifty microliters of this solution±antisense oligonucleotides (10⁻⁷ M) was added in the higher chamber of the modified Boyden chamber apparatus, and the lower chamber was filled with DMEM/1% FBS/antibiotics plus the proper concentration of agonist (VEGF or PlGF). The two sections of the system were separated by a porous polycarbonate filter (5 μm pores) pretreated with a gelatin solution (1.5 mg/ml), and assembled. Five hours post-incubation at 37° C., the non-migrated cells were scraped with a plastic policeman, the migrated cells were stained using Quick-Diff solutions. The filter was then mounted on a glass slide and migrated cells were counted using a microscope adapted to a video camera to obtain a computer-digitized image.

Measurement of PAF synthesis: PAF production by BAEC was measured by incorporation of ³H-acetate into lyso-PAF (Sirois, M. G., and Edelman, E. R. (1997) Am. J. Physiol 272, H2746-H2756). Confluent BAEC (100 mm tissue culture plate) were washed with DMEM and trypsinized. Cells were resuspended in DMEM/5% FBS/antibiotics, and a cell count was obtained. Cells were seeded at 5×10⁵ cells/well of 6 well tissue culture plates, stimulated for 24 h in DMEM/5% PBS/antibiotics±antisense oligonucleotides (10⁻⁷ M−5×107 M) and starved for 48 h in DMEM/0.25% FBS/antibiotics±antisense oligonucleotides (10⁻⁷ M−5×10⁻⁷ M daily) for Go synchronization. The cells were then grown to confluence for 24 h in DMEM/1% FBS/antibiotics±antisense oligonucleotides (10⁻⁷ M−5×10⁻⁷ M) and starved for 8 h in DMEM/0.25% FBS/antibiotics±antisense oligonucleotides (10⁻⁷ M−5×10⁻⁷ M) to induce an upregulation of VEGF receptor expression. Culture medium was removed and cells were rinsed twice with HBSS (Hank's balanced salt solution)/HEPES (10 mM; pH 7.4). Cells were then stimulated for 15 min in 1 ml of HBSS-HEPES (10 mM, pH 7.4)+CaCl₂ (10 mM)+³H-acetate (25 μCi) plus the appropriate concentration of agonist (VEGF or PlGP). The reaction was stopped by addition of acidified methanol (50 mM acetic acid), the wells were scraped and added to chloroform (2.5 ml) and 0.1 M sodium acetate (1 ml) mixture. Culture plates were washed twice with 1 ml of methanol, added to the chloroform mixture and centrifuged for 2 min at 1 700 rpm. The upper phase was discarded and the chloroform phase was washed twice with 2 ml of the organic phase of a HBSS-HEPES (10 mM)methanol-chloroform-sodium acetate (0.1M) solution (1:2.5:3.75:1). Isolated lipids were evaporated under a stream of N₂ gas, resuspended in 175 μl of mobile phase solvent (water-chloroform-methanol 5:40:55) and purified by HPLC. Samples were injected into a silica-based normal-phase BPLC column (4.5×250 mm, 5 μm silica particle size; Varian, Harbour City, Calif.) and eluted with the mobile phase solvent at a 0.5 ml/min flow rate. Fractions were collected every min and the amount of ³H-PAF synthesised was quantified by counting radioactivity with a β-counter. The authenticity of synthesized ³H-PAF was confirmed by an HPLC elution pattern similar to standard ³H-PAF (New England Nuclear), and by its ability to induce platelet aggregation similar to standard PAF (Avanti Polar Lipids, Alabaster, Ala.) (Sirois, M. G., and Edelman, E. R. (1997) Am. J. Physiol. 272, H2746-H2756).

Statistical Analysis: Data are mean±SEM. Statistical comparisons were made by analysis of variance followed by an unpaired Student's t-test. Data were considered significantly different if values of P<0.05 were observed.

Results

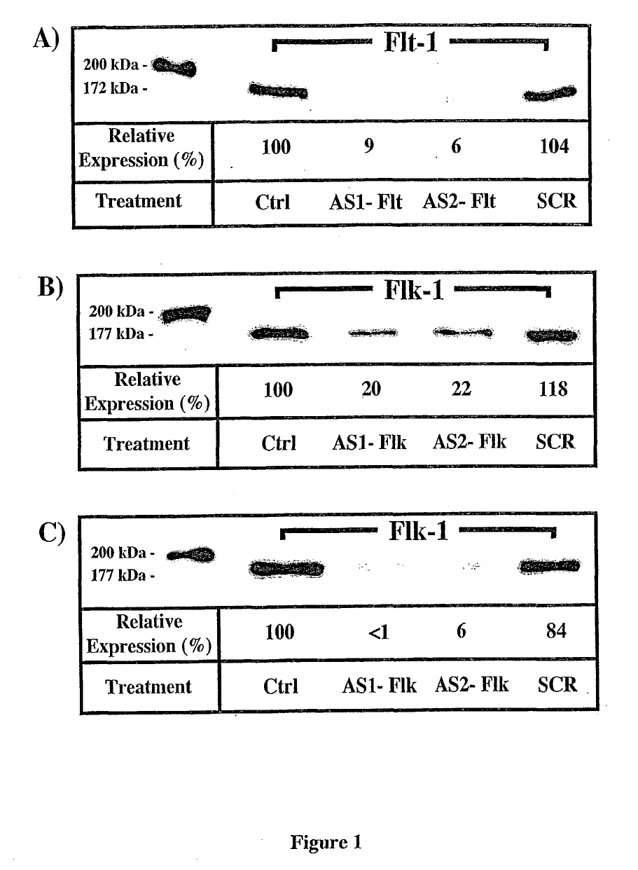

Modulation of Flt-1 or Flk-1 protein expression by antisense oligonucleotides: In order to determine the potency of antisense oligonucleotides to inhibit the targeted protein expression, BAEC were pretreated with either the antisense or the scrambled oligonucleotide sequences. Total proteins were extracted, quantified by bioassay, immunoprecipitated with an anti-Flt-1 or an anti-Flk-1 antibody, and the expression of each receptors was determined by Western blot analysis. Digital image densitometry was performed and results were expressed as relative expression percentages when compared with control PBS-treated cells. The basal protein expression of Flt-1 (Ctrl) was inhibited when the BAEC were pretreated with the two antisense complementary to Flt-1 mRNA (10⁻⁷ M); the first antisense sequence (AS1-Flt) suppressed Flt-1 protein expression by 91%, while the second antisense sequence (AS2-Flt) showed a 94% inhibition effect (FIG. 1A). Similar treatment with the two antisense sequences (ASI-bFlk-1 and AS2-bFlk-1; 10⁻⁷ M) complementary to Flk-1 mRNA suppressed basal FIk-i protein expression by 80% and 78%, respectively (FIG. 1B). Two scrambled sequences (SCR-Flt and SCR-Flk; 10⁻⁷ M) had no inhibitory effect on the studied receptor expression as compared to control cells (FIGS. 1A and B). To achieve a greater inhibition of Flk-1 protein expression, BAEC were pretreated with a higher concentration of antisense (AS1-bFlk and AS2-bFlk; 5×10⁻⁷ M), resulting in a 99% and 94% suppression of Flk-1 protein expression respectively (FIG. 1C). The scrambled sequence (5×10⁻⁷ M) showed a slight reduction by 16% of Flk-1 protein expression (FIG. 1C).

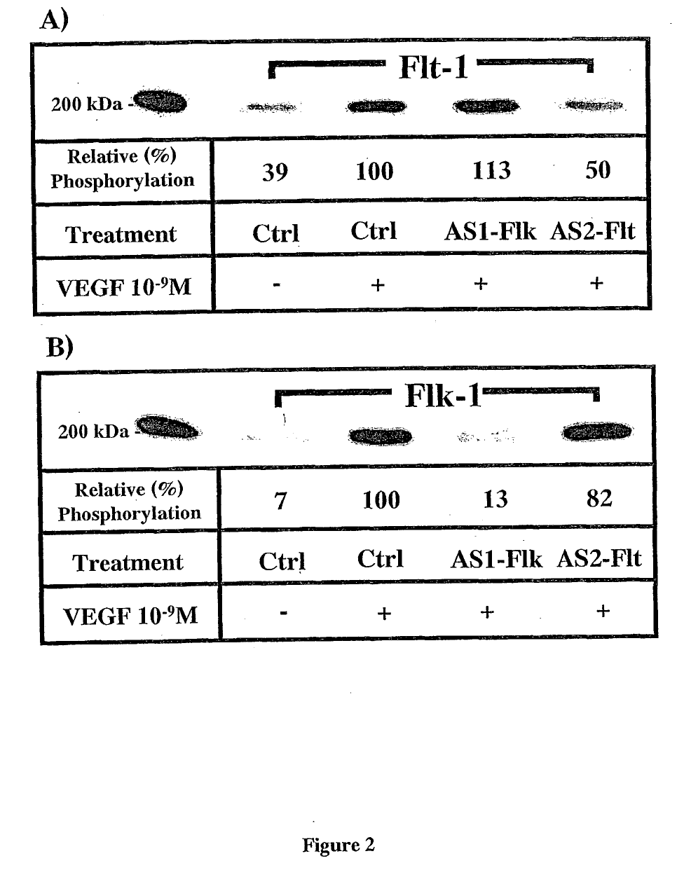

To ensure that the antisenses designed to downregulate the expression of Flk-1 would not affect Flt-1 receptor expression and vice versa, a Western blot analysis was performed to evaluate the specificity of our most potent antisenses. A pretreatment with the more potent antisense for the downregulation of Flk-1 expression (AS1-bFlk; 5×10-7 M) did not significantly affect Flt-1 basal expression (FIG. 2A) while the more potent antisense designed for the blockade of Flt-1 receptor expression (AS2-bFlt; 5×10⁻⁷ M) almost completely blocked Flt-1 receptor expression (FIG. 2A). A pretreatment with AS1-bFlk (5×10⁻⁷ M) severely impaired Flk-1 protein expression as compared to non-treated cells, while AS2-bFlt (5×10⁻⁷ M) was without significant effect (FIG. 2B).

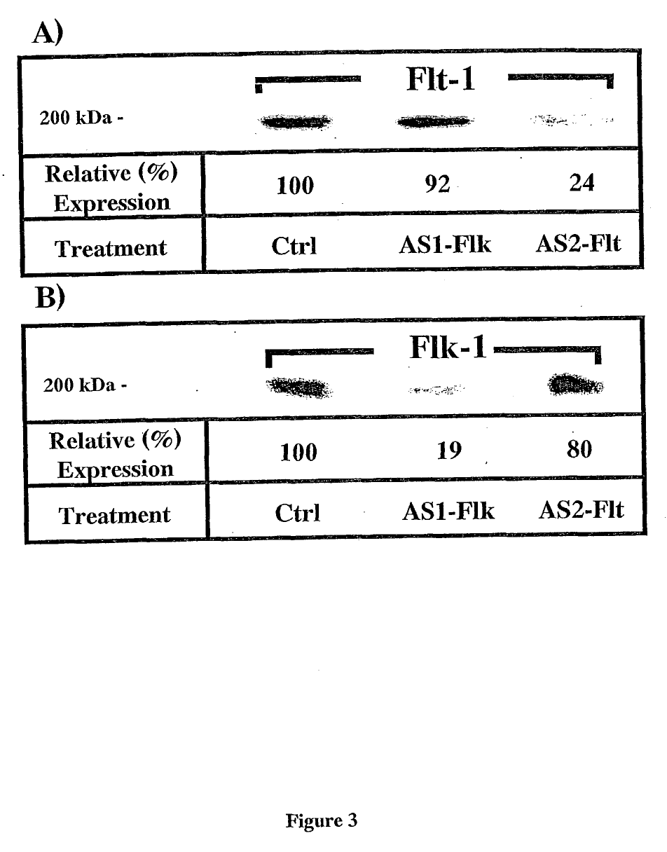

Inhibition of VEGF-induced Flt-1 or Flk-1 phosphorylation by antisense oligomers: Since the herein described antisense sequences were found to be specific at blocking the targeted receptor expression, it was then necessary to determine their potency to modulate Flt-1 and Flk-1 protein phosphorylation upon stimulation with VEGF. First, the stimulation of BAEC with VEGF (10⁻⁹ M) induced an increase of Flt-1 and Flk-1 phosphorylation by up to 1.5 and 13.2-fold respectively, over PBS-treated cells (FIGS. 3A and B). Pretreatment with the more potent antisense directed against Flt-1 mRNA (AS2-bFlt; 5×10⁻⁷ M) reduced by 50% the VEGF-induced phosphorylation of Flt-1 protein, while the more potent antisense directed against Flk-1 mRNA increased its phosphorylation by 13% (FIG. 3A). A similar pretreatment with the AS1-bFlk (5×10⁻⁷ M) inhibited by as much as 87% the phosphorylation of Flk-1 receptor (FIG. 3B), while a pretreatment with AS2-bFlt (5×10⁻⁷ M) slightly decreased Flk-1 phosphorylation in response to VEGF (10⁻⁹ M) by 18% (FIG. 3B).

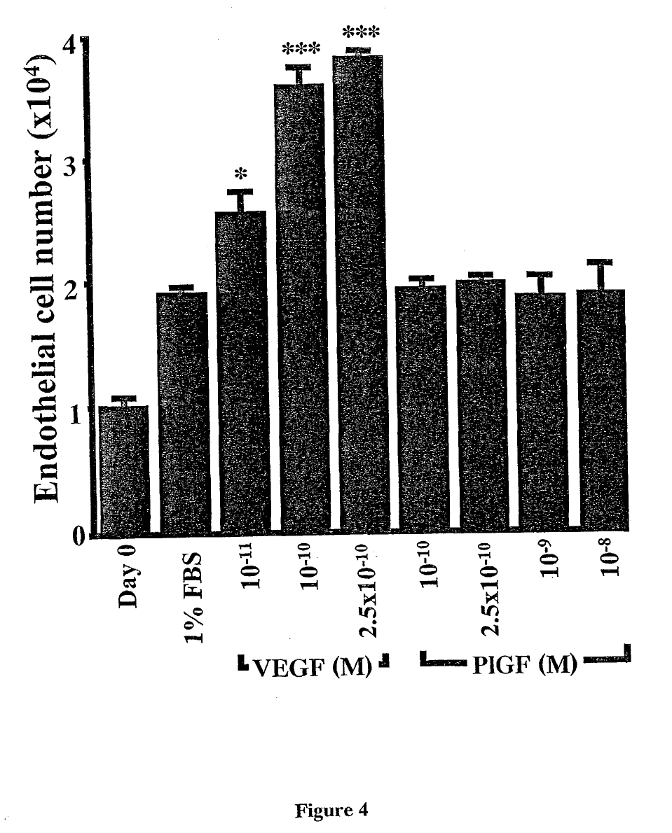

VEGF and PlGF mitogenic activity on BAEC: The VEGF and PlGF mitogenic effects were examined in order to discriminate the involvement of the two VEGF receptors on BAEC proliferation. Stimulation of quiescent BAEC with DMEM/1% FBS raised the cell count from 10 080±520 to 19 180±600 cells within 72 h. The addition of VEGF (10⁻¹¹, 10⁻¹⁰ and 2.5×10⁻¹⁰ M) increased endothelial cell proliferation dose-dependently with maximal induction of 62%, 183% and 219% respectively as compared to DMEM/1% FBS (FIG. 4). In contrast, PlGF (10⁻¹¹, 10⁻¹⁰, 10⁻⁹ and 10⁻⁸ M) did not show any mitogenic activity on BAEC as compared with DMEM/1% FBS (FIG. 4).

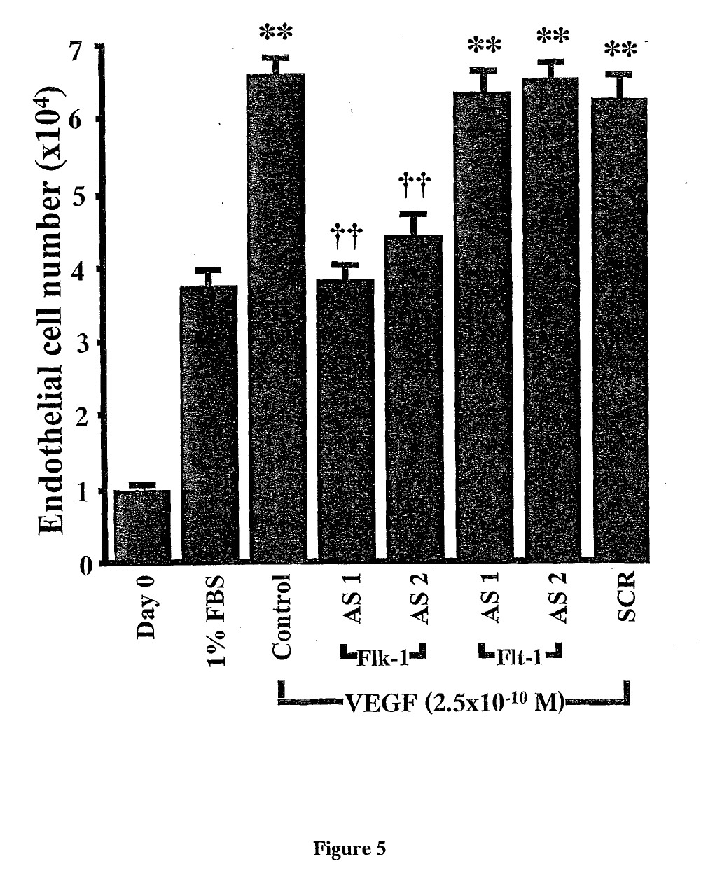

Effects of antiseise oligonucleotides complernentaty to Flk-1 and Flt-1 mRNA on VEGF mitogenic activity: By downregulating the protein expression of Flk-1 and Flt-1 by antisense gene targeting, it was possible to determine the contribution of each receptor type to VEGF's mitogenic effect on BAEC. FBS (1%) increased BAEC count from 9 860±640 to 37 260±2 260 cells. The addition of VEGF (2.5×10⁻¹⁰, M) increased BAEC proliferation by an additional 105% (P<0.01) (FIG. 5). Treatment of BAEC with the two antisense sequences directed against the Flk-1 mRNA completely blocked VEGF's mitogenic activity. The scrambled oligonucleotide sequences also failed to block VEGF-induced proliferation of BAEC.

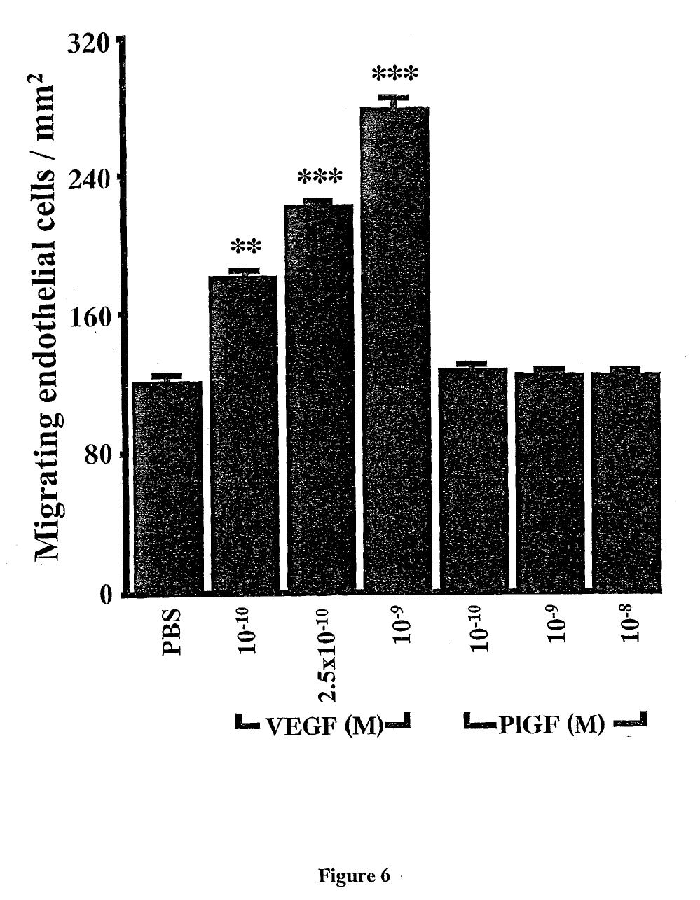

VEGF and PlGF chemotactic activity on BAEC: Using a modified Boyden chamber assay, the chemotactic response of BAEC to VEGF and PlGF was studied. VEGF (10⁻¹⁰, 2.5×10⁻¹⁰ and 10⁻⁹ M) induced a dose-dependent increase (46%, 83%, and 130% respectively) of BAEC migration as compared to PBS-stimulated cells, raising the migrated cell count from 120±4 (PBS) to 276±8 cells/mm² (VEGF 10⁻⁹ M; P<0.001) 5 hours post-treatment (FIG. 6). Checkerboard analysis revealed that the response of BAEC to VEGF was a result of chemotaxis and not chemokinesis. Treatment with PlGF (10⁻¹⁰, 10⁻⁹ and 10⁻⁸ M) had no significant effect on the basal migration of BAEC as compared to PBS-stimulated cells (FIG. 6).

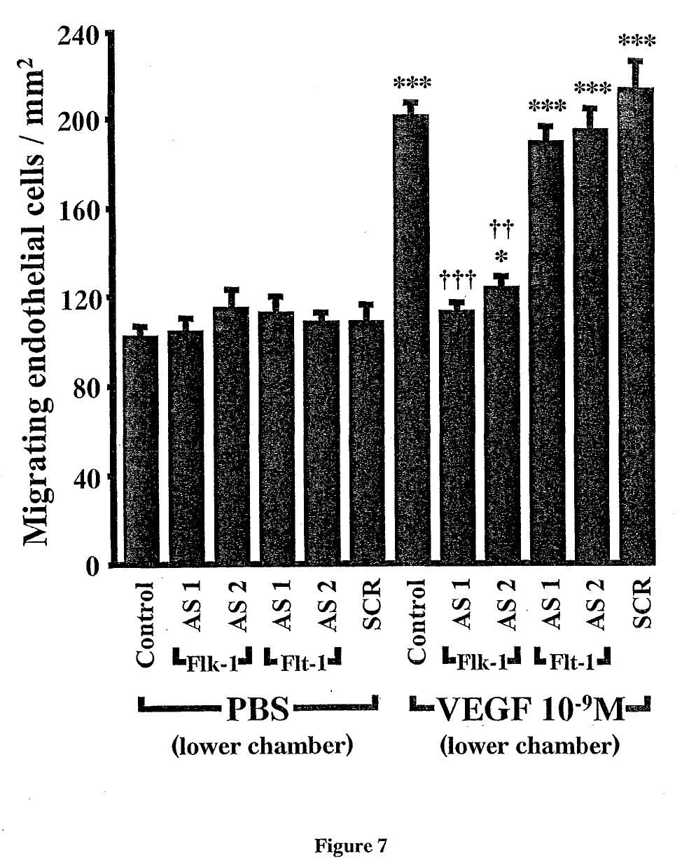

Effects of Flk-1 and Flt-1 mRNA antiseinse oligonucleotides on VEGF chemotactic activity: Non-stimulated BAEC (PBS) showed a basal migration count of 105±7 cells/mm² (FIG. 7). Stimulation with VEGF (10⁻⁹ M) increased the migrated cell count to 205±5 cells/mm². Pretreatment of BAEC with any of the four antisense sequences (AS1 or AS2-bFlk, AS1 or AS2-bFlt; 10⁻⁷ M) or scrambled sequences (SCR-Flt or SCR-Flk; 10⁻⁷ M) did not significantly affect basal migration in the absence of VEGF. In contrast, the antisense oligonucleotide sequences complementary to Flk-1 mRNA, AS1-bFlk and AS2-bFlk (10⁻⁷ M), decreased by 91% and 80% respectively the migration elicited by VEGF. The use of the antisense sequences to Flt-1 mRNA (10⁻⁷ M) did not alter VEGF-induced chemoattraction of BAEC. The scrambled oligonucleotide sequences did not significantly affect the chemotactic properties of VEGF (FIG. 7).

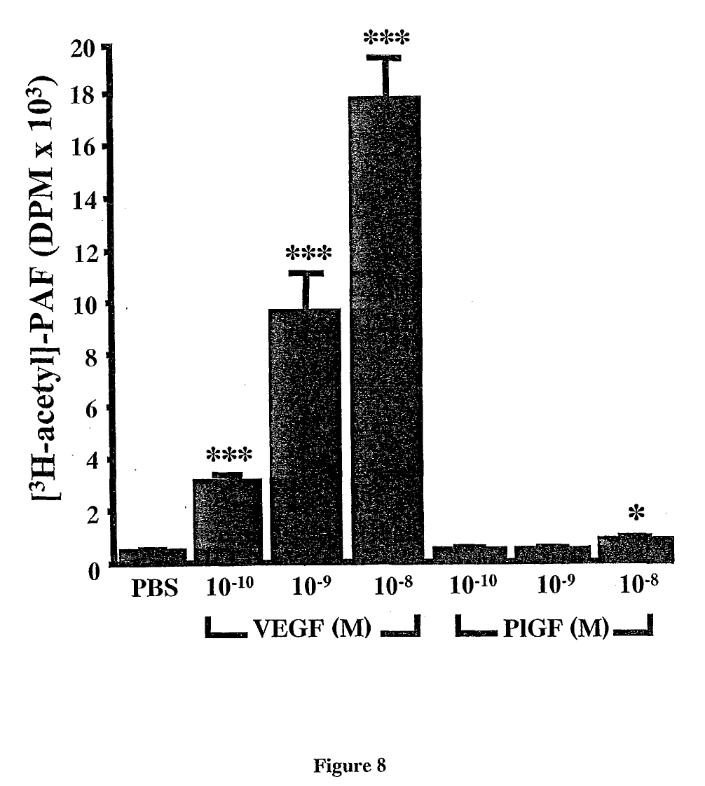

VEGF and PlGF effects on endothelial cell PAFsynthesis: To determine whether VEGF and PlGF stimulated PAF synthesis in EC, confluent BAEC were incubated with growth factors and PAF synthesis was determined by metabolic incorporation of ³H-acetate into lyso-PAF, the precursor of PAF synthesis. VEGF (10⁻¹⁰, 10⁻⁹ and 10⁻⁸ M) dose-dependenitly elicited the synthesis of PAF, with increases of 7.2-, 20.4- and 35.9-fold respectively as compared to PBS-treated cells (FIG. 8). Treatment with PlGF (10⁻¹⁰, 10⁻⁹ M) did not significantly affect the basal PAF synthesis of BAEC. However, at 10⁻⁸ M, PlGF induced a slight but significant increase in PAF synthesis (67%) as compared to PBS-treated cells (FIG. 8).

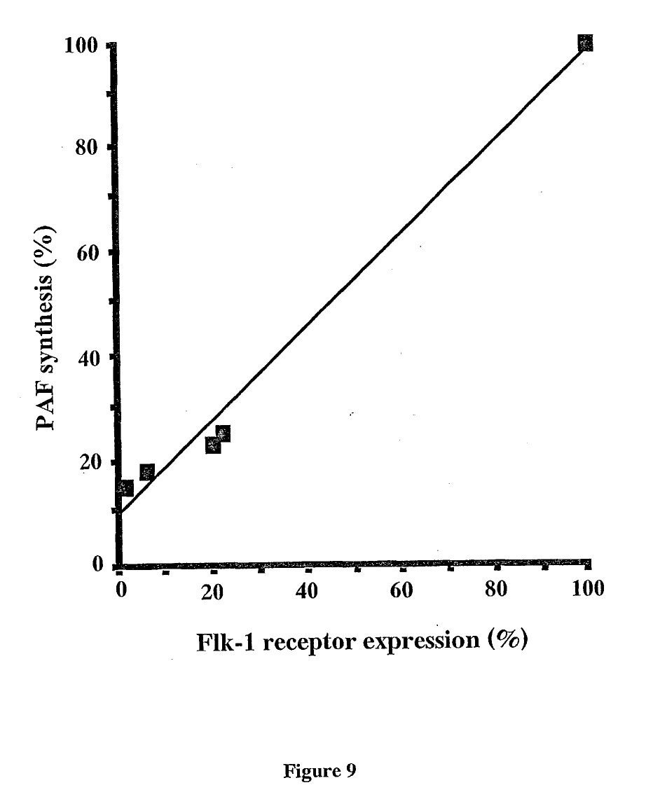

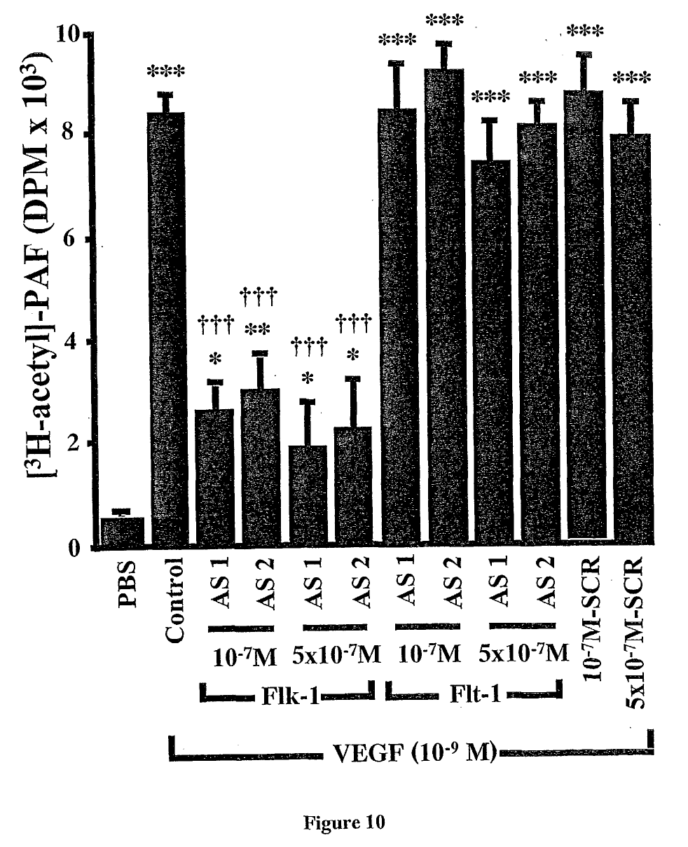

Effects of Flk-1 and Flt-1 mRNA antisense oligonucleotides on VEGF-induced PAF synthesis: In order to determine the basal and maximal PAF synthesis by BAEC, a group of cells were left untreated and others were treated with VEGF (10⁻⁹ M) for 15 minutes. The synthesis of ³H-labelled PAF increased from 781±86 to 8 254±292 DPM (FIG. 9). Treatment of BAEC with the antisense oligonucleotide sequences complementary to Flk-1 mRNA, AS1-Flk and AS2-Flk, (10⁻⁷ M) reduced by 77% and 75% respectively the synthesis of PAF elicited by a VEGF treatment (FIG. 9). In contrast, pretreatment with the antisense oligonucleotide sequences complementary to Flt-1 mRNA (10⁻⁷ M) failed to inhibit VEGF's inflammatory activity on BAEC. The scrambled oligonucleotide sequences (SCR; 10⁻⁷ M) also failed to affect VEGF-induced PAF synthesis (FIG. 9). Since both antisense oligonucleotide sequences complementary to Flk-1 mRNA (10⁻⁷ M) failed to fully inhibit PAF synthesis induced by VEGF (10⁻⁹ M), the concentration of antisense directed against Flk-1 mRNA was increased to 5×10⁻⁷ M during BAEC treatment. The application of AS1-Flk and AS2-Flk (5×10⁻⁷ M) caused a near complete inhibition of Flk-1 protein expression (FIG. 1C) and a reduction of PAF synthesis by 85% and 82% respectively in response to VEGF (10⁻⁹ M), while the two antisense sequences complementary to Flt-1 mRNA (5×10⁻⁷ M) did not inhibit VEGF-induced PAF synthesis (FIG. 9). The absence of nonspecific inhibitory effects was furthermore confirmed by pretreating BAEC with the scrambled sequences (5×10⁻⁷ M) which did not affect PAF synthesis. As the inhibition of Flk-1 expression had a direct effect on PAP synthesis, a correlation analysis was performed. The synthesis of PAF by BAEC treated with VEGF (10⁻⁹ M) showed a linear correlation increment with Flk-1 protein expression [PAP synthiesis %=n×Flk-1 expression %+b], where m is the slope and b is the linearity constant. Our data showed a slope m of 0.89 and a linear constant b of 9.81 (r²=0.984; FIG. 10).

Discussion

Angiogenesis is a tightly regulated process, integral to normal and pathological conditions. Crucial steps in the angiogenic process support an early increase in vascular permeability (Dvorak, H. F., et at (1995) Am. J. Pathol. 146, 1029-1039), closely followed by migration and proliferation of EC. Much evidence implicates VEGF and its two tyrosine kinase receptors Flt-1 and Flk-1 as major regulators of these events (Waltenberger, J., et al. (1994) J. Biol. Chem. 269, 26988-26995; Brown, L. F., et al (1995) Human Pathol. 26, 86-91; Ravindrath, N., et al (1992) Endocrinology 94, 1192-1199; and Breier, G., et al. (1992) Development 114, 521-532). VEGF, unlike any other growth factors studied to date, is capable of inducing protein extravasation and it is likely that its angiogenic properties are mediated in large part through the induction of plasma protein leakage (Dvorak, H. F., et al (1995) Am. J. Pathol. 146, 1029-1039). It was recently shown that VEGF's effect on vascular permeability was mediated through the synthesis of PAF by EC (Sirois, M. G., and Edelman, E. R. (1997) Am. J. Physiol 272, H2746-H2756).

The present invention demonstrates that the proliferation, migration and PAF synthesis elicited by VEGF in cultured BAEC are dose-dependent (FIGS. 4, 6 and 8) and above all, these effects were completely (proliferation) or almost completely (migration and PAF synthesis) inhibited by treating the cells with specific antisense oligonucleotide sequences complementary to FPk-1 receptor mRNA.

Antisense oligomers specifically inhibit Flt-1 or Flk-1 receptor expression.

Both Flt-1 and Flk-1 are cell surface-associated receptors deemed to play a role in VEGF-induced EC activation. Recent studies have investigated their signal transduction properties using porcine aortic endothelial cells or NIH 3T3 cells transfected with a plasmid coding either for Flk-1 or Flt-1 (Waltenberger, J., et al (1994) J. Biol. Chem. 269, 26988-26995; Seetharam, L., et al (1995) Oncogene 10, 135-147). Recently, many novel VEGF-related molecules (PlGF, VEGF-C, VEGF-C-ΔNΔC156S mutant) which vary in their potency to activate one of the two VEGF receptors preferentially were isolated and characterized (Park, J. E., et al (1994) J. Biol. Chem. 269, 25646-25654; Joukov, V., et al (1998) J. Biol. Chem. 273, 6599-6602; and Clauss, M., et al (1996) J. Biol. Chem. 271, 17629-17634). Although the use of these analogs suggested that both receptors could mediate biological actions, they do not assess the possible formation of heterodimers, which has been proposed to occur between VEGF receptors when signaling (Waltenberger, J., et al (1994) J. Biol. Chem. 269, 26988-26995).

In the present invention antisense gene therapy was used to suppress specifically the Flt-1 and Flk-1 gene products. This approach allowed the use of fresh non-transfected endothelial cells which endogenously express the two VEGF receptors and the intracellular pathways found in native EC. In addition, since it was possible to inhibit separately the Flt-1 and Flk-1 protein expression, the present system provided the possibility to evaluate if Flt-1 and Flk-1 heterodimerization was required to observe the VEGF biological activity.

This example made use of two selective antisense oligonucleotide sequences for the Flt-1 receptor mRNA, and two others for the Flk-1 receptor mRNA. These sequences did not contain more than three consecutive guanosines to avoid a possible interference with serum proteins including growth factors like VEGF (Stein, C. A. (1995) Nature Med. 1, 1119-1121). Having the assurance that BAEC express both VEGF receptors (Pepper, M. S., et al (1998) J. Cell. Physiol. 177, 439-452), the ability of antisense oligomers to specifically inhibit the expression and phosphorylation patterns of Flt-1 and Flk-1 was determined. As shown by Western blot analysis, BAEC expressed Flt-1 and Flk-1 proteins (FIGS. 1A, B and C) which were both phosphorylated by a VEGF treatment (FIGS. 3A and B). Treatment of BAEC with the antisense Flt-1 oligomers (up to 5×10M) for a 4 day period decreased the protein expression of Flt-1 receptor by as much as 94% (AS2-bFlt; FIG. 1A) and inhibited its phosphorylation by up to 50% in response to a VEGF stimulation (10⁻⁹ M; FIG. 3A). Treatment with the antisense Flk-1 oligomers (10⁻⁷ M) was also effective at modulating Flk-1 receptor expression, with a maximum inhibition of 80% (AS1-bFlk). The difference in the inhibitory percentage is in accordance with previous reports which showed that the biological effects of antisense oligomers are dictated in part by the kinetics of antisense target gene expression (Edelman, E. R., et al (1995) Circ. Res. 76, 176-182). The difference between Flk-1 and Flt-1 oligomers was overcome by increasing the antisense Flk-1 oligomer concentration to 5×10⁻⁷ M, resulting in a greater reduction in the residual Flk-1 expression when compared with the 10⁻⁷ M treatment, from a 80% to a 99% inhibition of Flk-1 protein expression. This latter pretreatment also prevented VEGF-induced FPk-1 protein phosphorylation by as much as 87% (FIG. 3B).

Previous reports have raised concerns that the inhibitory activities of antisense oligonucleotides may arise from non-specific rather than hybridization-dependent mechanisms (Burgess, T. L., et al (1995) Proc. Natl. Acad. Sci. U.S.A. 92, 4051-4055; and Guvakova, M. A., et al (1995) J. Biol. Chem. 270, 2620-2627). To address this issue more definitely, two groups of BAEC were pretreated with two different scrambled oligomers at similar concentrations (10⁻⁷−5×10⁻⁷ M). In contrast to the VEGF receptor antisense oligomers, the scrambled oligomers (10⁻⁷ M) failed to modulate the normal pattern of VEGF receptors protein expression by BAEC, although it showed a slight reduction at a higher concentration (5×10⁻⁷ M). In addition, no cross-reactivity was observed between the Flk-1-directed antisense sequences and Flt-1 expression and vice versa (FIGS. 2A and B). It is to be noted also that the scrambled oligomers (10⁻⁷−5×10⁻⁷ M) did not inhibit VEGF effect on EC proliferation, migration and PAP synthesis.

Antisense Oligomer-Directed Modulation of VEGF Activities

Since the antisense sequences used in this example specifically prevented both the protein expression and phosphorylation of Flt-1 or FIk-1 genes, they were tested for their ability to modulate VEGF properties on EC. A treatment with AS1-Flk (10⁻⁷ M) was sufficient to provide a complete inhibition of VEGF mitogenic effect (FIG. 5), and abolished almost completely (91% inhibition) the cellular migration induced by VEGF (FIG. 7). However, this approach inhibited by 75% the synthesis of PAF (FIG. 9). A higher concentration of AS1-Flk (5×10⁻⁷ M) induced not only a higher inhibition of Flk-1 protein expression (FIG. 1B), but also blocked the PAP synthesis elicited by VEGF by as much as 85% (FIG. 9). This demonstration suggests that Flk-1 plays a role in mediating VEGF effects on BAEC. The correlation between the synthesis of PAF from BAEC stimulated with VEGF (10⁻⁹M) and the expressed Flk-1 receptors on these EC was also demonstrated. A linear correlation was established and suggested that a complete inhibition of Flk-1 protein expression by antisense oligomers against Flk-1 mRNA would still permit VEGF to induce a 9.8% residual PAF synthesis by treated BAEC (FIG. 10). Such a minor effect can possibly be explained either by Western blot analysis limitation to fully detect residual Flk-1 protein expression or by a partial contribution of Flt-1 stimulation. Though a 99% inhibition of Flk-1 protein expression was observed, it is possible that more than 1% of Flk-1 receptors were still present on BAEC surface, which could not be detected by either the immunoprecipitation process or the protein revelation by chemiluminescence after a Western blot study. The other possibility to explain the residual PAF synthesis may involve a partial effect through the activation of Flt-1 receptors. This latter hypothesis is supported by the data from PlGF treatment of BAEC.

PlGF is a secreted growth factor expressed by umbilical vein EC and placenta (Maglione, D., et al (1991) Proc. Natl. Acad. Sci. U.S.A. 88, 9267−9271; and Hauser, S., and Weich, H. (1993) Growth Factors 9, 259-268). According to its amino acid sequence, PlGF shows a partial homology to VEGF (53% homology), which might explain its ability to bind uniquely to Flt-1 (Park, J. E., et al (1994) J. Biol. Chem. 269, 25646-25654; and Clauss, M., et al (1996) J. Biol. Chem. 271, 17629-17634). Therefore, PlGF can be used to study the effect of Flt-1 activation on EC. Although various concentrations of PlGF (10⁻¹⁰-10⁻⁸ M) failed to elicit EC proliferation and migration, PlGF at 10⁻⁸ M induced a slight but significant increment of PAF synthesis over control levels, suggesting that Flt-1 may indeed participate in mediating PlGF and VEGF action on EC. This is in agreement with previous reports which have shown that Flt-1 stimulation either by PlGF or VEGF can induce Flt-1 phosphorylation (Waltenberger, J., et al (1994) J. Biol. Chem. 269, 26988-26995; Cunningham, S. A., et al (1997) Biochem. Biophys. Res. Commun. 240, 635-639; and Sawano, A., et al (1997) Biochem. Biophys. Res. Commun. 238, 487−491). However, the biological activities mediated by either VEGF or PlGF upon Flt-1 activation/phosphorylation on intracellular Ca²⁺ elevation, cellular proliferation, migration and procoagulant tissue factor production observed were either absent or weak (Waltenberger, J., et al (1994) J. Biol. Chem. 269, 26988-26995; Clauss, M et al (1996) J. Biol. Chem. 271, 17629-17634; Hauser, S., and Weich, H. (1993) Growth Factors 9, 259-268; Cunningham, S. A., et al (1999) Am. J. Physiol. 276, C176-C181) as compared to Flk-1 activation/phosphorylation. Consequently, the residual PAF synthesis (10%) that was observed following an antisense Flk-1 oligomer treatment as estimated by the linear correlation may in fact be due to: 1) an incomplete suppression of the Flk-1 protein expression and/or 2) from intracellular signaling through Flt-1 receptor activation. In addition, VEGF may interact with Flt-1 differently than PlGF and induce a greater PAF synthesis. Therefore, these data support the hypothesis that Flt-1 stimulation is capable of mediating biological response, but to a lower extent than Flk-1 stimulation.

VEGF, Flt-1 and Flk-1

Many studies suggest that VEGF and its two receptors may take part in the angiogenesis phenomenon. For instance, homozygous disruption of the Flk-1 gene leads to embryonic death due to failure of vasculogenesis whereas homozygous Flt-1 disruption allows normal vascular endothelial differentiation and development but leads to a failure to assemble normal vascular channels and death (Fong, G. H., et al (1995) Nature 376, 66-70; and Sharrna, H. S., et al (1992) Exper. Suppl 61, 255-260). In this example, the inhibition of Flk-1 protein expression severely impaired VEGF effects on EC, which supports the importance of this receptor for VEGF activity.

In summary, this example demonstrates that antisense oligomer-directed inhibition of Flk-1 receptor expression severely impaired VEGF-induced EC proliferation, migration and PAF synthesis.

### Example II

### VEGF-Mediated Angiogenesis-Role of Flk-1 AND Flt-1

Receptors

Material and Methods

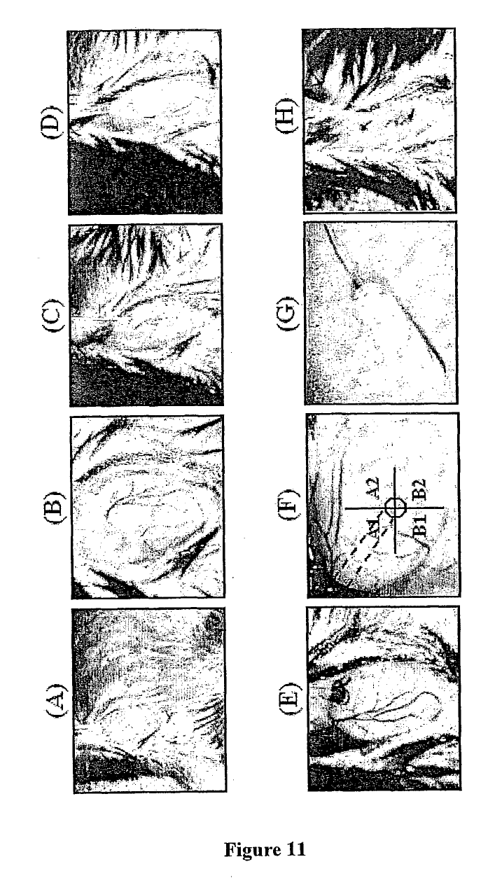

Surgical procedures: The surgical procedures were performed by one trained operator and in accordance to the guidelines set by the Montreal Heart Institute animal care committee and the Canadian Council for Animal Protection. Male mice C57/B16 (weight, 18-22 g) (Charles River Breeding Laboratories, Saint-Constant, QC) were anesthetized with an intraperitoneal injection of ketamine HCl 100 mg/kg (Ketalean, MTC Pharmaceuticals, Cambridge, ON) and xylazine HCl 10 mg/kg (Rompun, Bayer, Etobicoke, ON).A diagonal incision (2 cm) of the skin was made just above the right groin upon disinfection of the skin with chlorexidine (0.5%, Novopharm, Toronto, ON). The rectus abdominis muscle and transversalis fascia were dissected to get access to the peritoneal cavity (FIG. 11A). The right testis was pulled out through the inguinal canal and brought to the skin incision (FIG. 11B). A fine needle 25G5/8 was used to create a micropuncture in the visceral layer of the tunica vaginalis of the testis, near the head of the epididymis where there were no apparent vessels (FIG. 11C). A sterilized PEIO catheter (Cole-Parner Instrument Company, Vernon Hills, Ill.) was introduced into the testis in a selected area and secured with silk 6-0 (Davis & Geck, Wayne, N.J.) attached to the tunica vaginalis (FIGS. 11D-E). The testis was repositioned into the scrotum by passing through the inguinal canal, and the rectus sheath was sutured with silk 6-0 (FIG. 11H). The free tip of the catheter inserted in the testis was fixed with silk 6-0 to the rectus sheath to prevent unwanted movements and connected to a larger catheter PE60 (Becton Dickinson and Company, Sparks, Md.). This latter was adapted to a mini-osmotic pump (2002, Alza Corporation, Palo Alto, Calif.) with a controled flow delivery of 0.5 μl/hour; 14 days (FIG. 1G). The pump was placed subcutaneously, on the abdominal right flank. The wound was then closed with dexon 5-0 (Davis & Geck, Wayne, N.J.) and the animals were returned to their cages.

The mini-osmotic pumps were pre-filled with 200 μl of PBS-BSA (0.1%) (Sigma Chemical Co., St-Louis, Mo.), VEGF (Pepro Tech inc., Rocky Hill, N.J.) at different concentrations (1, 2.5, 5 μg/200 μl PBS-BSA 0.1%) to obtain a dose-response curve on the induction of blood vessel formation. Based on these data (see details in results section), a group of mice was treated with VEGF (2.5 μg/100 μl PBS-BSA 0.1%) combined to AS-Flk-1 (200 μg/100 μl PBS-BSA 0.1%), AS-Flt-1 (200 μg/100 μl PBS-BSA 0.1%) or AS-scrambled (200 μg/100 μl PBS-BSA 0.1%). Another group of mice was treated with the oligoiners (200 μg/200 μL PBS-BSA 0.1%) in absence of VEGF.

Finally, a sham operated group of animals was performed, the testes were manipulated as above, without the insertion of catheter and osmotic pump. After 14 days of treatment, the animals were anesthetized and dissected as described above in order to bring the right testis to the skin incision for image acquisitions. Animals were then sacrificed using an overdose of ketamine and xylazine.

Image acquisitions and analysis: Pictures of various regions of the testis with inserted catheters were taken at different magnifications (8.4×, 12×, 24×, 38.4×, 48×) with a color video digital camera (Sony DKC 5000) adapted to a binocular (Olympus SZX12). To assess the number of new blood vessels, the surface of the testis was divided into 4 sections: A1, A2, B1 and B2 (FIG. 11F). For each testis, one picture per section was taken at day 0. Then a picture of the exact same region was taken at day 14 after treatment. These pictures were taken at a magnification of 48×, and the pictures at day 0 and day 14 were then compared. The number of new blood vessels present at day 14.but absent at day 0 was counted on each picture for each section. The new blood vessels counted were full-length vessels of at least 150 μm and not the result of sprouting. The surface of the pictures taken at 48× magnification was 1.288 mm², and the number of new blood vessels was converted as the number of new blood vessels per mm² by dividing the number of new blood vessels per field of 48× by the surface (1.288).

In order to confirm that the new blood vessels observed at day 14 were not pre-existing blood vessels that have been vasodilated by a VEGF treatment, we did another set of experiments in which pictures were taken at day 0 and 14 (in VEGF treated groups), then, the Alzet mini-osmotic pump was removed and a new set of pictures were taken 3 days later (day 17) in such, the VEGF effect was no longer involved as VEGF has a plasmatic half life of 3 minutes (Folkman J., (1995) Nat Med 1: 27−31).

The images taken before (day 0) and after treatment (day 14 and in some cases at day 17) were then compared and different parameters were determined: number of new blood vessels, length and diameter of the new vessels, change in the diameter of pre-existing vessels and immunohistochemistry analysis. The number of new blood vessels was determined by counting directly on the pictures the number of new vessels created by various treatments. The length and diameter of the vessels were calculated by computerized digital planimetry with a dedicated video binocular and customized software (NIH image 1.6).

Selection of the antisense oligomers: The antisense oligonucleotides were selected and designed in function of specific characteristics such as no more than three consecutive guanosines, the incapacity to form hairpins and a minimal capacity to dimerize together, and the length of the antisense oligonucleotides is generally between 15 to 25 bases. The murine Flt-1 and Flk-1 cDNA were obtained from GENBANK (GenBank Accession Numbers D28498 and X70842) respectively. A total of four different antisense oligonucleotide phosphorothioate backbone sequences were selected, two targeting mice Flt-1 mRNA (AS1-mFlt: 5′-AAG CAG ACA CCC GAG CAG-3′ (SEQ ID NO:5); AS2-mFlt: 5′-CCC TGA GCC ATA TCC TGT-3′ (SEQ ID NO:6)), and two targeting mice Flk-1 mRNA (AS1-mFlk: 5′-AGA ACC ACA GAG CGA CAG-3′ (SEQ ID NO:7); AS2-mFlk: 5′-AGT ATG TCT TTC TGT GTG-3′ (SEQ ID NO:8).

Two scrambled phosphorothioate sequences (scrambled Flt, SCR2-Flt: 5′-ACT GTC CAC TCG CAG TTC-3′ (SEQ ID NO:21); scrambled Flk, SCR2-Flk: 5′-TTT CTG GTA TGC ATT GTG-3′ (SEQ ID NO:22)) were also selected as negative controls. All sequences were synthesised at the Armand Frappier Institute (Laval, Canada). After synthesis, the oligonucleotides were dried, resuspended in sterile PBS, filtered (0.2 μm pore size) and quantified by spectrophotometry. The assurance that the antisense oligomer solutions were by-products-free will be confirmed by denaturing polyacrylamide gel electrophoresis (20%; 7M urea), based on the known length of the oligonucleotide.

Immunohistochemistry of Flk-1, Flt-1 and ecNOS expression: After sacrifice of the animals, testes were isolated, fixed in 10% formalin PBS-buffered solution and processed for standard histological procedures. Testes sections were cut into 6 μm longitudinal sections, deparaffinized in xylene and ethanol baths, endogenous peroxidase activity was quenched in a solution of methanol (200 ml) plus hydrogen peroxide (30%, 50 ml), nonspecific binding of primary antibodies was prevented by preincubating the tissues with serum 5% from the species used to raise the secondary antibodies. Testes sections were then exposed to primary antibodies for 1 hr (ecNOS) or 2 hrs (Flk-1 and Flt-1). The primary antibodies used were monoclonal anti-mouse Flk-1 IgG (Santa Cruz Biotechnology Inc., Santa Cruz, Calif.) diluted (1:500, 1 000, 2 500), rabbit polyclonal anti-human Flt-1 IgG (Santa Cruz Biotechnology Inc., Santa Cruz, Calif.) diluted (1:100, 250, 500), and monoclonal anti-human endothelial cell constitutive nitric oxide synthase (ecNOS) IgG (Transduction Laboratories, Mississauga, ON) diluted (1:2 500, 5 000, 10 000). Purified non-specific mouse IgG (for Flk-1 and ecNOS detection) or rabbit IgG (for Flt-1) were used as primary negative control antibodies. Upon incubation, the primary antibodies were washed with PBS, the slides incubated 60 minutes either with a biotinylated goat anti-rabbit (for Flt-1 detection) or a goat anti-mouse IgG (for Flk-1 and ecNOS detection) (1:400) (Vector Labs Inc., Burlingame, Calif.). Peroxidase labelling was achieved with an incubation using avidin/peroxidase complex (ABC kit; Vector Labs Inc.), and antibody visualization established after a 5 minute exposure to 3,3′-diaminobenzidine solution (DAB kit; Vector Labs Inc.). Testes were counterstained by Gill's hematoxylin #3 solution, rinsed in tap and distilled water and mounted with a permount solution.

Statistical analysis: Data are mean±SEM. Statistical comparisons were determined by ANOVA followed by a paired or unpaired Student's t test with Bonferroni's correction for multiple comparisons. Data were considered significantly different if a value of P<0.05 was observed.

Results

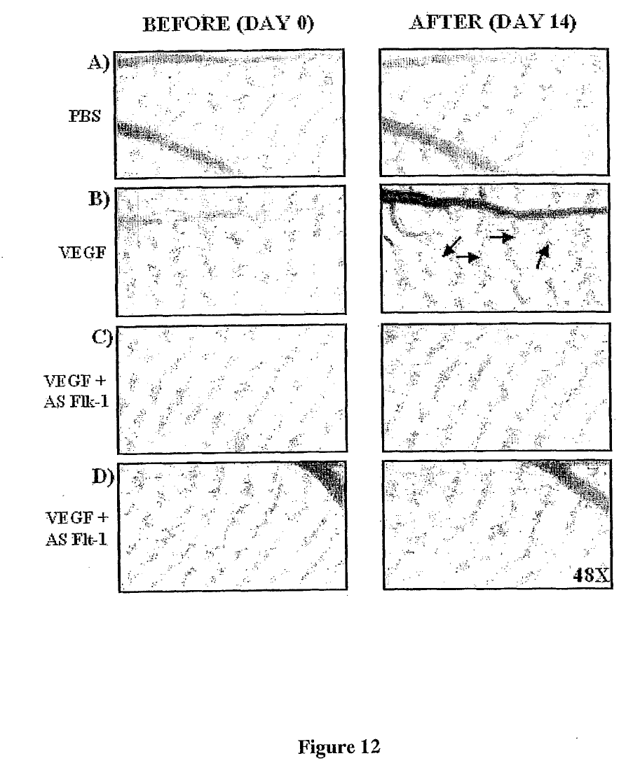

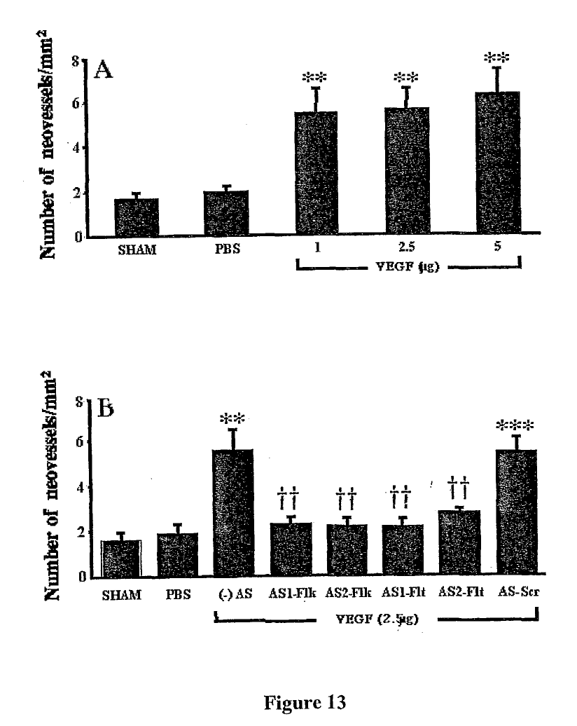

Angiogenesis assessment: The infusion of PBS (200 μl) on a 14-day period with a mini-osmotic pump adapted to a catheter inserted in the testis induced the formation of 1.86±0.37 new blood vessels/mm² (FIGS. 12A and 13A). This formation of new blood vessels was not different from the one observed in control sham operated animals 1.58±0.27 new blood vessels (FIG. 13A). Treatment with VEGF at different concentrations (1, 2.5 and 5 μg/200 ill) delivered on a 14-day period increased significantly the number of new blood vessels by 236 (P<0.01), 246 (P<0.01) and 287% (P<0.01) respectively as compared to sham control groups (FIGS. 12B and 13A).

Effect of AS on the formation of new blood vessels: Based on the data presented in FIG. 13A, VEGF was used at a dose of 2.5 μg for the following experiments. As mentioned above, the infusion of VEGF (2.5 μg/200 μl) for a period of 14 days induced the formation of 5.48±0.96 new blood vessels/mm² (P<0.01; as compared to sham control group) (FIG. 3B). The combination of AS1-mFlk-1 (200 μg), AS2-mFlk-1 (200 μg), AS1-mFlt-1 (200 μg) or AS2-mFlt-1 (200 μg) to VEGF (2.5 μg) into 200 μl (final volume) decreased the formation of new blood vessels by 85, 87, 85 and 71% respectively as compared to VEGF treated group (P<0.01) (FIGS. 2C-D and 3B). The combination of scrambled oligomers (AS-Scr; 200 μg) to VEGF (2.5 μg) into 200 μl (final volume) led to the formation of 5.34±0.64 new blood vessels/mm² which was not statistically different from the group treated with VFGF alone (FIG. 13B). In another group the effect of the antisense and scrambled oligomers in PBS treated mice was tested. These oligomers did not alter significantly 1) the number of pre-existing blood vessels and 2) the basal formation of new blood vessels mediated by PBS (data not shown).

The length and the diameter of the new blood vessels were also determined. The average length of the new blood vessels in all studied groups fluctuated from 245 to 324 μm. The average length of new blood vessels under VEGF treatment (2.5 μg/200 μl) was 284±10 μm (Table 1). The diameter of the new blood vessels was also measured and all had a capillary-like diameter with an average diameter fluctuating from 6.30 to 9.04 μm, including an average diameter of 8.52±0.40 μm under VEGF treatment (2.5 μg/200 μl) (Table 1).

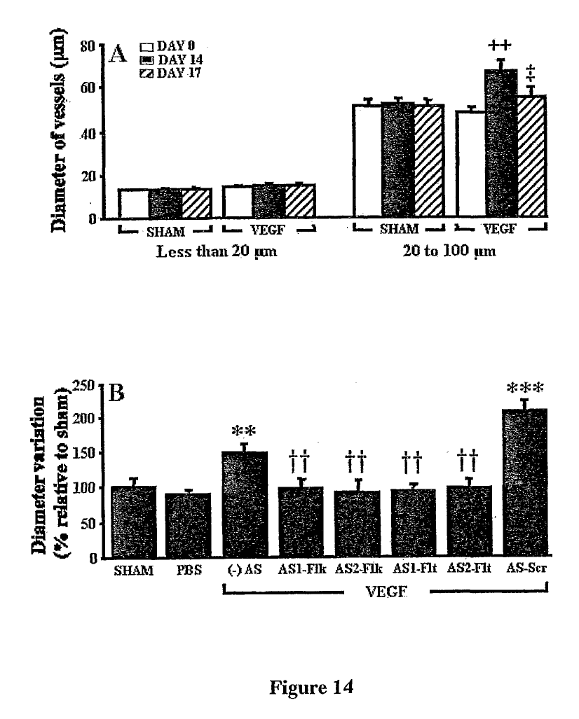

Vasodilatory effect of VEGF on pre-existing blood vessels: VEGF is a vasodilatory mediator, consequently, and it was assessed whether the new blood vessels observed upon a sustained infusion of VEGF were due to the dilation of pre-existing capillaries or due to its angiogenic potential. The vasodilatory effect of VEGF was studied on pre-existing blood vessels with a diameter smaller than 20 μm and on vessels with a diameter between 20 to 100 μm. Pictures of the testes to be treated were taken at day 0 before treatment and at day 14, then the mini-osmotic pump was removed and another set of pictures taken 3 days later at day 17. In the control sham-operated group, there was no change in the diameter of pre-existing vessels at day 14 and 17 as compared to the diameter observed at day 0 (FIG. 14A). A treatment with VEGF (2.5 μg/200 μl) delivered on a period of 14 days did not mediate a vasodilation of pre-existing vessels with a diameter smaller than 20 μm. The diameter of these vessels increased by 6% at day 14 compared to day 0 and decreased by 3% at day 17 compared to day 14 (P=NS) (FIG. 14A). However, the infusion of VEGF (2.5 μ/200 μl) on a period of 14 days increased by 40% the dilation of pre-existing blood vessels having a diameter between 20 to 100 μm as compared to untreated arteries (day 0) (P<0.01). However, this vasodilatory effect mediated by VEGF infusion was abrogated within a period of 3 days following the arrest of VEGF infusion (day 17) and was no longer significant as compared to day 0 (FIG. 14A).

Effect of AS on VEGF-mediated vasodilation of pre-existing blood vessels: In sham-operated mice, the diameter of pre-existing blood vessels (20 to 100 μm of diameter) did not fluctuate significantly from day 0 to day 14, and the mean diameter was set as the 100% baseline diameter. A treatment with VEGF (2.5 μg/200 μl) delivered on a 14 days period induced the vasodilation of pre-existing blood vessels (20 to 100 1 μm of diameter) by 48% (P<0.01 as compared to control sham operated mice) (FIG. 14B). The combination of AS1-mFlk-1 (200 fig), AS2-mFlk-1 (200 μg), AS1-mFlt-1 (200 μg) or AS2-mFlt-1 (200 μg) to VEGF (2.5 μg) into 200 μl (final volume) abrogated the VEGF-vasodilatory effect of pre-existing blood vessels (20 to 100 μm of diameter) (P<0.01) (FIG. 14B). Treatment with a scrambled oligomer did not reduce the VEGF-mediated vasodilatory effect. In fact, it even increased the vasodilation of pre-existing blood vessels (FIG. 4B). The combination of antisense or scrambled oligomers to PBS did not alter the basal diameter of the pre-existing blood vessels (20 to 100 μm of diameter) (data not shown).

Effect of VEGF on the number and diameter of new blood vessels: In the previous studies (above) it was demonstrated that under sustained VEGF infusion the presence of new blood vessels occurred within 14 days and they were less than 10 μm of diameter. In addition, it was observed that VEGF infusion had no vasodilatory effect on pre-existing blood vessels with a diameter below 20 μm, but induced the vasodilation of pre-existing blood vessels with a diameter above 20 μm, and this effect was abrogated upon the arrest of VEGF infusion (FIG. 14A). These results suggest that the new blood vessels observed cannot be the result of a vasodilation of pre-existing blood vessels with a diameter below 20 μm of diameter as VEGF does not induce vasodilation of these small blood vessels.

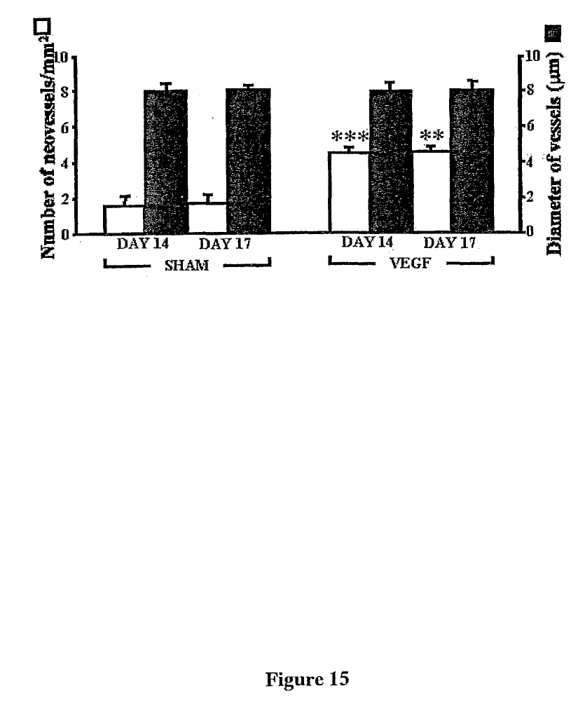

Nevertheless, to ensure that the new blood vessels observed under VEGF infusion were not the result of an unexpected vasodilation of small pre-existing capillaries (<10 μm diameter), an additional study was performed in which the number and the diameter of new blood vessels at day 14 and 17 was quantitated in control sham and VEGF-treated mice (FIG. 15). In control sham operated mice, the formation of 1.71±0.40 new blood vessels/mm² with a mean diameter of 8.01±0.40 μm was observed at day 14, these parameters were not significantly different at day 17 (FIG. 15). A treatment with VEGF (2.5 μg/200 μl) infused on a 14-day period induced in the present study the formation of new blood vessels (4.40±0.40 vessels/mm²) (P<0.001) as compared to a control group, and these new vessels had a diameter of 7.92±0.47 μm, which is not statistically different from the diameter of new blood vessels formed in control sham-operated group (FIG. 15). The infusion of VEGF was terminated by the removal of the mini-osmotic pump at day 14, and the same parameters were evaluated 3 days later (day 17) (FIG. 15). The number and diameter of new blood vessels 3 days upon the removal of VEGF were not statistically different from those obtained at day 14 under VEGF treatment (FIG. 15).

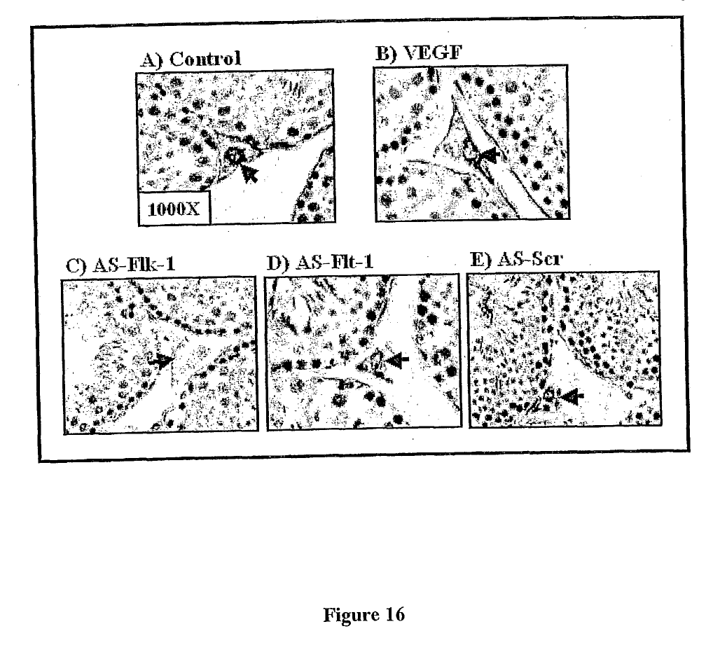

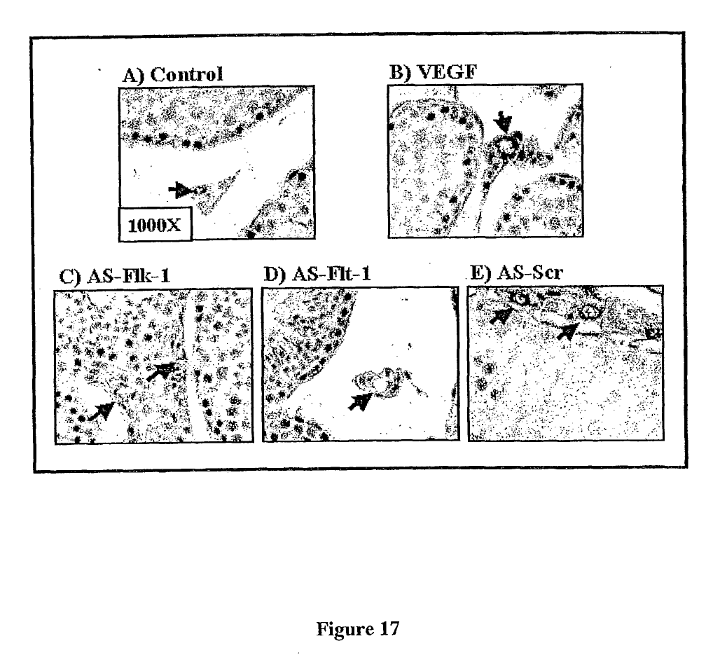

Effect of AS and VEGF on Flk-1 and Flt-1 protein expression: A semi-quantitative analysis of Flk-1 and Flt-1 protein expression was performed using immunohistochemical analysis. The expression of Flk-1 and Flt-1 receptors is present on vascular endothelial cells of mouse testes under normal condition (non-treated, sham operated or PBS-infused) (FIGS. 16A and 17A). Under VEGF sustained infusion (2.5 μg/200 μl; 14 days period), it was observed that the protein expression of mFlk-1 and mFlt-1 remained similar to control sham operated group as compared to control sham-operated group (FIGS. 16B and 17B). It was then investigated whether the effect of AS1-mFlk-1 (200 μg), AS2-mFlk-1 (200 μg), AS1-mFlt-1 (200 μg) or AS2-mFlt-1 (200 μg) combined with VEGF (2.5 μg) in 200 μl (final volume). A treatment with either AS1-mFlk-1 or AS2-mFlk-1 blocked mFlk-1 protein expression (FIG. 16C), without affecting mFlt-1 protein expression (FIG. 17C), whereas a treatment with either AS1-mFlt-1 or AS2-mFlt-1 blocked mFlt-1 protein expression (FIG. 17D), without affecting mFlk-1 protein expression (FIG. 16D). A treatment with scrambled oligomers did not alter the expression of mFlk-1 and mFlt-1 (FIGS. 16E and 17E). Purified non-specific mouse and rabbit IgG were used as primary negative control antibody, and in each case no positive staining was detected (data not shown).

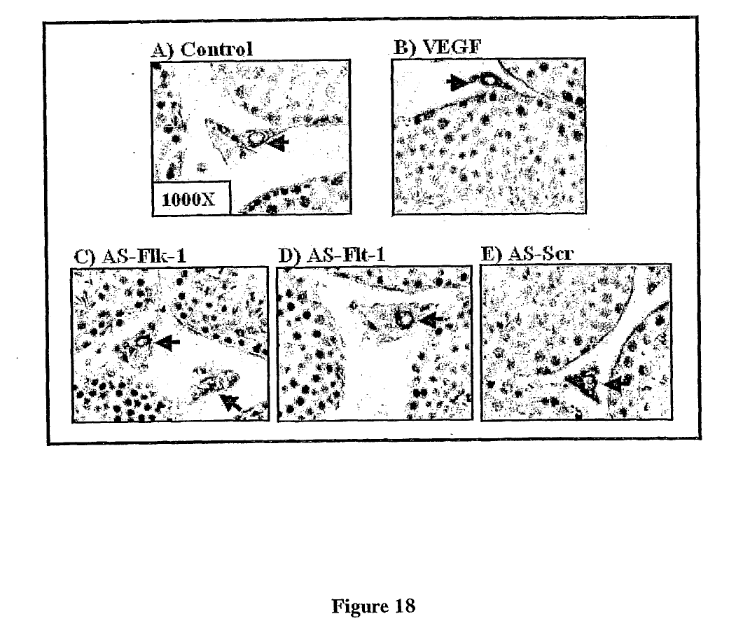

Endothelial cell nitric oxyde synthase (ecNOS) protein expression: VEGF mediated a vasodilation of pre-existing blood vessels with a diameter between 20 to 100 μm, and such vasodilation was abrogated by antisense oligomers targeting either mFlk-1 or mFlt-1 mRNA but not with scrambled oligomers (FIG. 14B). It was then important to confirm that the inhibition of VEGF-mediated vasodilation by the antisense oligomers was not due to a non-selective downregulation of ecNOS protein expression. Using immunohistochemistry analysis the ecNOS protein expression on vascular endothelial cells of mouse testis under normal conditions (non-treated, sham operated or PBS-infused) and in VEGF-treated group (FIGS. 18A and 18B) was demonstrated. The combination of antisense oligomers either against mFlk-1 or mFlt-1 mRNA as well as scrambled oligomers with VEGF did not alter the ecNOS protein expression on vascular endotlielial cells of mouse testis (FIGS. 18C-E). Purified non-specific mouse IgG was used as primary negative control antibody, and no positive staining was detected (data not shown).

Discussion

The present example provides a new model of angiogenesis in which enables a skilled worker to: 1) investigate VEGF angiogenic activity, 2) down-regulate by antisense oligonucleotides gene therapy the protein expression of Flk-1 and Flt-1, 3) prevent VEGF-mediated angiogenesis, and 4) demonstrate that Flt-1 and Flk-1 are required to mediate VEGF vasodilatory effect.

The angiogenic model used in the present example offers several advantages over those most often used in laboratories. It provides the possibility to work with mammalian animal. The testis has a moderate vascular network at the surface of the tunica vaginalis which is very easy to locate and to measure. Consequently, it is also very easy to see the formation of new capillary-like blood vessels under angiogenic conditions, and the results are highly reproducible. The surgical procedure is relatively easy, and produces no inflammatory response at the expected angiogenic site. Furthermore, as the testis is pulled out through the natural inguinal canal for the surgical procedure and for image acquisitions, there is consequently no scar tissue of fibrosis formation on the testis. In this model, drugs and mediators of interest can be delivered locally in the testis through a catheter adapted to a mini-osmotic pump placed distally. This latter approach provides a significant advantage as it allows (if desired) to modify the treatment by the removal/replacement of the delivering mini-osmotic pump upon a simple skin incision at the level of the abdominal flank, and thus, without having to handle the treated testis. If applicable, the angiogenic inhibitors can be given by other routes (orally, intravenously etc).

In the present example it was demonstrated that a sustained infusion for 14 days of VEGF in mouse testis induced the formation of new capillary-like blood vessels with a normal blood flow. In addition, it was demonstrated that treatment with antisense oligonucleotides directed against the mRNA of VEGF receptors Flk-1 or Flt-1 for a period of 14 days abrogated almost completely the VEGF-mediated angiogenesis. These results show that both VEGF receptors Flk-1 and Flt-1 are essential for VEGF in vivo angiogenic activity.

As demonstrated herein VEGF can induce a vasodilation of blood vessels with a diameter of 20 to 100 μm, such vasodilation was not observed in microvessels with a diameter inferior to 20 μm. This latter effect can be explained by the fact that these vessels (<20 μm diameter) are composed mainly of a monolayer of endothelial cells with no or sparse smooth muscle cells surrounding them which would provide the capacity to modulate the vascular tone. What is even more interesting is the fact that a treatment with the antisense oligonucleotides against the mRNA of VEGF receptors Flk-1 or Flt-1 inhibited the vasodilation of pre-existing blood vessels (20 to 100 μm diameter) mediated by VEGF, whereas scrambled oligomers had no such effect. This result shows that both VEGF receptors Flk-1 and Flt-1 are essential for VEGF vasodilatory effect.

Using immunohistochemical analysis on testes sections, it was confirmed that the antisense oligonucleotides against the mRNA of Flk-1 and Flt-1 reduced selectively their corresponding protein expression, whereas the scrambled oligomers did not alter the Flk-1 and Flt-1 protein expression. Nevertheless, as the use of antisense oligomers targeting either Flk-1 or Flt-1 mRNA abrogated VEGF-vasodilatory effect and since VEGF may mediate the release of NO, an ecNOS immunohistochemnical analysis was performed to ensure that the antisense oligomers did not directly or indirecty alter ecNOS protein expression. This study confirmed the ecNOS protein expression on testes vasculature of control and VEGF treated-mice, and that neither the antisense oligomers targeting Flt-1 or Flk-1 mRNA nor scrambled oligomers altered the ecNOS protein expression. These data confirm that the inhibition of VEGF-induced vasodilation and angiogenesis is not caused by the inhibition of vascular ecNOS protein expression.

To rule-out the possibility that that the new blood vessels observed upon a 14 day VEGF infusion was not due to the vasodilation of pre-existing capillaries that could not be seen at day 0 prior to VEGF treatment, VEGF was infused for 14 days, then removed the mini-osmotic pump, and collected additional images 3 days later (day 17). At day 14, new blood vessels were observed having a diameter below 10 μm and pre-existing blood vessels with a diameter between 20 to 100 μm were observed to be vasodilated as compared to the diameter observed at day 0. Three days after the arrest of VEGF infusion (day 17), the vasodilatory effect of VEGF on preexisting blood vessels (20 to 100 μm of diameter) could no longer be observed, but the new blood vessels observed at day 14 were still present at day 17 and their diameter was maintained (below 10 μm). These data confirm that the new blood vessels observed at day 14 were the result of VEGF-angiogenic activity in mouse testis.

In summary, the present study introduces a convenient and reproducible model which allows the investigation in vivo angiogenesis. Antisense oligonucleotide based gene therapy was shown to downregulate the protein expression of Flk-1 and Flt-1, and in both cases, abrogate VEGF angiogenic activity. Therefore, there results demonstrate that the blockade of VEGF receptors expression by antisense gene therapy provides a new therapeutic approach to prevent diseases associated with pathological angiogenesis.

### Example III

### Flk-1 and Flt-1 VEGF receptors activationis Essential to Hyperoxia-Induced Retinopathy

Selective Blockade of Flt-1 and Flk-1 VEGF Receptor Expression Prevent Hyperoxic Retinopathy in Newborn Mice.

Interventions targeting VEGF synthesis have suggested the involvement of this cytokine in retinal angiogenic responses caused by hyperoxia, however the role of the various VEGF receptor subtypes in this process is not known. Antisense oligonucleotides can targeted directed against specific VEGF receptors to determine which VEGF receptor subtype is involved in pathological hyperoxia-induced retinopathy. By blocking the effects of VEGF derived from both local (ocular) and extraocular sources, the intraocular antisense-induced downregulation of VEGF receptors offers benefits over the less specific conventional approaches.

Animals: Seven day (D7) old mouse pups and their nursing mothers (C57/BL6 wild type) will be exposed for 5 days to hyperoxic conditions (75% O₂) with 4 daily 30 minutes periods of normoxic conditions. After 5 days (D12), mice will be returned to nornoxic conditions for an additional 5 days at which time, maximal retinal neovascularization is observed (D17). This leads to a reproducible and quantifiable oxygen-induced retinopathy, as demonstrated earlier (Heller R, et al, (1992) J. Immunol. 149: 3682-3688; Fujikawa K, et al, (1999) Exp. Cell Res. 253: 663-672; White P. (1960) Diabetes 9: 345-355; Rand L. I., (1981) Am. J. Med. 70: 595-602.). Under anesthesia, drugs will be injected in the vitreous with a 32-gauge Hamilton needle syringe. Each eye will receive a bolus of 0.5 μl. Because the volume of the vitreous is estimated at 50 μl, injected drugs will be diluted by 100 times. Up to 3 injections can be performed at different sites over the duration of the experiments.

Protocols: Seven day (D7) old mouse pups and their nursing mother will be exposed to the hyperoxic conditions and returned to normoxia as described above. Mice will be treated with intraocular injection of antisense oligomers at day 4 (D11) of the hyperoxic condition, the day after (D13) and the third day (D15) after the return to normoxia. The antisense oligomers targeting the mRNA of selected mouse VEGF receptors will be as follow: AS-Flt-1: 5′-AAG CAG ACA CCC GAG CAG-3′; AS-Flk-1: 5′-AGA ACC ACA GAG CGA CAG-3′. Two scrambled (SCR) phosphorothioate sequences (SCR-Flt-1: 5′-ACT GTC CAC TCG CAG TTC-3′; SCR-Flk-1: 5′-TTT CTG GTA TGC ATT GTG-3′) will be used. The efficacy of these antisense sequences in preventing VEGF-receptor expression and induction of angiogenesis in the mouse testes has been demonstrated.

The list below indicates the experimental groups involved. Each group will include mice maintained in normoxia throughout the duration of the experiment and mice undergoing the hyperoxia/nornoxia protocol:

1) Sham injected animals (insertion of the needle+PBS-vehicle infusion);

2) Scrambled-Flk-1 (10 μg/0.5 μl; final concentration in the vitreous 50 μM);

3) Scrambled-Flt-1 (10 μg/0.5 μl; final concentration in the vitreous 50 μM);

4) Antisense-Flk-1 (10 μg/0.5 μl; final concentration in the vitreous 50 μM);

5) Antisense-Flt-1 (10 μg/0.5 μl; final concentration in the vitreous 50 μM);

6) Scrambled oligomers Flt-1+Flk-1 (5 μg each/0.5 μl; final concentration 50 μM)

7) Antisense oligomers Flt-1+Flk-1 (5 μg each/0.5 μl; final concentration 50 μM)

About 28 pregnant mice will be required to yield the 8-10 pups per group that is required for statistical analysis. (7 sets of experiments×2 groups=14 groups×10 pups=140 pups; ≈5 pups per litre).

Determination of retinal surface vascularization. After enucleation, the eyes from each mouse will be fixed in 4% paraforinaldehyde in cacodylate buffer (0.1M, pH 7.2) and store in Tris buffer (50 mM). Interior eye structures and vitreous will be removed gently. Retinal vessels and neovascular buds, will be revealed by adenosine diphosphate histochemistry using the lead phosphate technique (Penn J. S., et al. (1994) Investig Ophthalmol Vis Sci 35:3429; Zhang S., (2000) Investigative ophtalmo. Visual Sci. 41: 887−891) which is equivalent in sensitivity to trans-sectional histology to detect neovascular tufts (Smith L. E. H, et al, (1999) Nature Med. 5: 1390-1395). Measurement of peripheral avascular areas is determined by highlighting vasculature by binary transformation of tonality (Adobe Photoshop) and tracing of the areas processed by digital imaging (NIH 1.6) (Zhang S., (2000) Investigative ophtalmo. Visual Sci. 41: 887−891).

Retinal histochemistry. The experiments detailed above will be duplicated for histochemical and immunohistochemical analyses. Following treatment, the eyes will be formalin-fixed, dehydrated and paraffin-embedded. Serial sections (6 μm) of the eyes will be cut sagitally parallel to the optic nerve and stained with Masson's-Trichrome solution. Extraretinal neovascularization will be assessed by counting the number of nuclei from the blood vessels extending into the vitreous beyond the inner limiting membrane of the retina. Multiple sections from each eye will be scored in a masked fashion by light microscopy adapted to a video camera-to obtain a computer-digitized image. The extend of neovascularization in the treated and control eyes will be determined by counting neovascular cell nuclei extending through the internal limiting membrane into the vitreous. The length and diameter of the new blood vessels will be quantified (Hardy P., et al (1998) Ophtalmol. Vis. Sci. 39: 1888-1898; Lachapelle P., et al (1999) Can. J. Physiol. Pharmacol. 77: 48-55; Nandgaonkar B N, et al (1999) Ped. Res. 46: 184-188.).

Retinal immunohistochemistry. The expression level of VECTF receptors (Flt-1 and Flk-1), and PCNA will be confirmed by immunohistochemistry as described earlier. In non-treated animals, the level of expression of VEGF receptors caused by hyperoxic conditions will be verified. In antisense-treated animals, this will allow to demonstrate the efficacy and selectivity of the therapy in limiting the expression of each VEGF receptor subtype. Proliferating vascular cells will be quantified by PCNA staining.

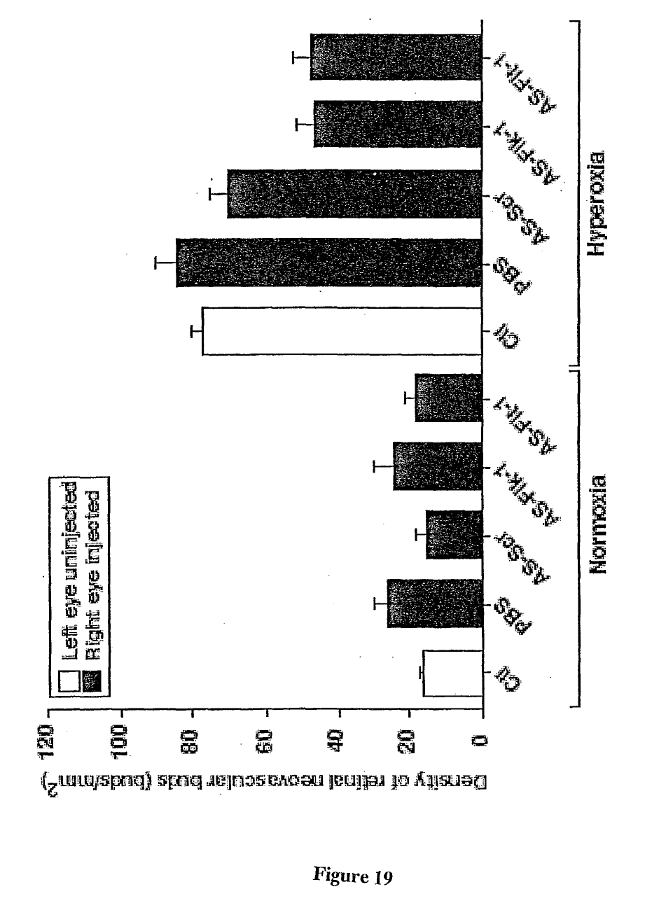

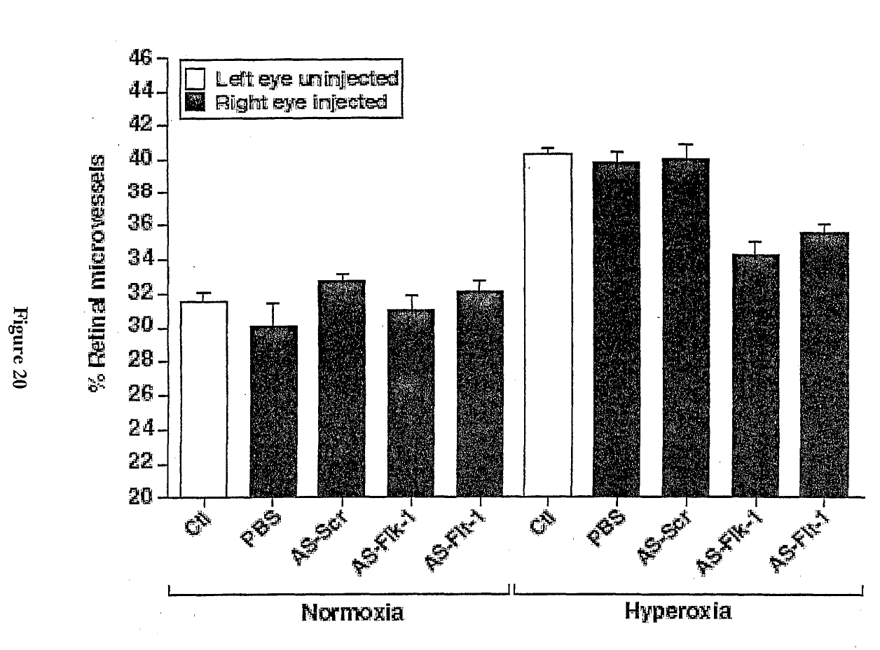

As shown in FIGS. 19 and 20, the antisense oligonucleotides targeting Flk-1 and Flt-1 mRNA (AS-Flk-1 and AS-Flt-1) (n=6 per group) reduced the retinal neovascularization mediated by hyperoxic treatment by 60 and 45%, and the budding of retinal neovessels by 58 and 57%. But did not affect basal retinal neovascularization and budding of retinal neovessels under normoxic condition. It is likely that the combined blockade of Flt-1 and Flk-1 receptor expression would further increase the inhibition of retinal angiopathy media by hyperoxic treatment.

### Example IV

### PAF is Essential to Hyperoxia-Induced Retinopathy

Inflammation is closely associated with the angiogenic process. It has been demonstrated that VEGF triggers the endothelial synthesis of a powerful inflammatory mediator namely, platelet-activating factor (PAF), and that a PAF receptor antagonist prevents VEGF inflammatory effect. It is also known that down-regulation of Flk-1 but not Flt-1 protein expression by antisense oligonucleotide application onto cultured endothelial cells selectively prevented VEGF-induced PAF synthesis.

PAF Activity is Essential to VEGF-Induced Angiogenic Activity.

The mouse testis model will be used to demonstrate that PAF is an essential mediator of the angiogenic activity of VEGF. It has been shown that intratesticular administration of VEGF increases capillary density (<10 μm, o.d.)>250%. Three treatment groups will be used to show the effect of PAF blockade on VEGF angiogenic activity: 1) VEGF±PAF receptor antagonist(s) Z) PAF±PAF receptor antagonist(s) 3) PAF receptor antagonist(s). Briefly, the inguinal canal is opened to isolate the right testis; a PE-10 catheter is inserted through the tunicae vaginalis and positioned in the testis. The other catheter end is connected to a subcutaneously placed Alzet pump 2002 for a sustained 14 day delivery period of VEGF, PAF, and/or a PAF antagonist. Angiogenesis is quantified by counting newly formed vessels visualized in situ with a microscopic videoimaging system before and at the end of drug delivery. In addition, testis will be processed for vascular morphometric analyses, and specific immunohistochemistry staining.

VEGF-Induced cGMP Production: Role of PAF

A variety of VEGF actions, including proliferation, migration, PAP synthesis and inflammatory response, may all be involved in the angiogenic response of this cytokine. Data not shown indicated that on cultured endothelial cells these VEGF-mediated effects involve phospholipase C-_{γ} and ras-dependent signalling pathways.

Flk-1 and Flt-1 VEGF Receptor Activation and PAF Synthesis are Essential to Hyperoxia-Induced Retinopathy.

A proliferative retinopathy model will further demonstrate the contribution of Flk-1 and Flt-1 receptor activation and PAF synthesis to pathological angiogenesis. Briefly, 7 day old mouse pups with their nursing mother will be exposed to hyperoxic conditions (75% O₂) for 5 days, leading to a reproducible and quantifiable angiogenic retinopathy. The mice will then be returned to room air, and under anesthesia, Flt-1, Flk-1 or scrambled antisense oligomers will be injected into the vitreous or a PAF-antagonist will be injected daily (i.p.). The animals will be sacrificed 5 days later and retinal vascularization analysed as described above.

These experiments will identify the pathways involved in the co-ordinated actions of VEGF on cultured endothelial cells. The in vivo angiogenesis project will link VEGF and PAF activity in the induction of angiogenesis, and the contributions delineated of VEGP receptor subtypes Flk-1 and Flt-1, and of PAF receptor activation in the process leading to pathological angiogenesis. These data provide the basis for future therapeutic strategies designed to inhibit pathological angiogenesis.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

TABLE 1
Vessel density, length and diameter of new blood
vessels according to treatment

| | new blood | | | |
|---|---|---|---|---|
| Treatment | vessels/mm² | length (μm) | diameter (μm) | n |
| Sham | 1.58 ± 0.27 | 251.2 ± 15.5 | 7.12 ± 0.26 | 6 |
| PBS | 1.86 ± 0.37 | 324.1 ± 17.0 | 6.62 ± 0.66 | 11 |
| VEGF (2.5 μg) | 5.48 ± 0.96 | 284.3 ± 10.0 | 8.52 ± 0.40 | 8 |
| AS1-Flk-1 | 2.17 ± 0.36 | 278.7 ± 13.1 | 7.74 ± 0.42 | 7 |
| A52-Flk-1 | 2.08 ± 0.40 | 278.9 ± 13.0 | 6.30 ± 0.59 | 7 |
| AS1-Flt-1 | 2.15 ± 0.40 | 285.2 ± 13.3 | 9.04 ± 0.44 | 8 |
| AS2-Flt-1 | 2.71 ± 0.23 | 267.4 ± 13.5 | 8.45 ± 0.48 | 7 |
| AS-scrambled | 5.34 ± 0.64 | 245.1 ± 6.4 | 7.94 ± 0.28 | 7 |

n = number of animals treated per group.

I claim:

1. An antisense oligonucleotide complementary to a gene encoding a mammalian vascular endothelial growth factor (VEGF) receptor selected from the group comprising Flt-1 and Flk-1, wherein said antisense oligonucleotide comprises about 15 to about 25 nucleotides complementary to said gene and wherein said VEGF receptor is a non-bovine receptor.

2. The antisense oligonucleotide according to claim 1, wherein said mammalian VEGF receptor is Flt-1.

3. The antisense oligonucleotide according to claim 1, wherein said mammalian VEGF receptor is Flk-1.

4. The antisense oligonucleotide according to claim 2, wherein said mammalian VEGF receptor is bovine Flt-1.

5. The antisense oligonucleotide according to claim 2, wherein said mammalian VEGF receptor is murine Flt-1.

6. The antisense oligonucleotide according to claim 2, wherein said mammalian VEGF receptor is human Flt-1.

7. The antisense oligonucleotide according to claim 3, wherein said mammalian VEGF receptor is bovine Flk-1.

8. The antisense oligonucleotide according to claim 3, wherein said mammalian VEGF receptor is murine Flk-1.

9. The antisense oligonucleotide according to claim 3, wherein said mammalian VEGF receptor is human Flk-1.

10. A pharmaceutical composition comprising a pharmaceutically acceptable diluent and an antisense oligonucleotide complementary to a gene encoding a mammalian vascular endothelial growth factor (VEGF) receptor selected from the group comprising Flt-1 and Flk-1, wherein said antisense oligonucleotide comprises about 15 to 20 nucleotides complementary to said gene.

11. A method of reducing pathological angiogenesis in a mammal in need of such therapy, comprising the step of administering to said mammal the antisense oligonucleotide of claim 1.

12. A method of reducing pathological angiogenesis in a mammal in need of such therapy, comprising the step of administering to said mammal the pharmaceutical composition of claim 11.

13. A method of reducing platelet activating factor (PAF) synthesis in a mammal in need of such therapy, comprising the step of administering to said mammal the pharmaceutical composition comprising the antisense oligonucleotide of claim 3.

14. An antisense oligonucleotide complementary to a gene encoding a mammalian vascular endothelial growth factor (VEGF) receptor selected from the group comprising Flt-1 and FPk-1, wherein said antisense oligonucleotide comprises about 15 to about 25 nucleotides complementary to said gene.
